(19) United States
(12) Patent Application Publication
(10) Pub. No.: US 2023/0103407 A1
(43) Pub. Date: Apr. 6, 2023

(54) MATERIALS AND METHODS FOR THE TREATMENT OF GAUCHER DISEASE

(71) Applicants: University of Cincinnati, Cincinnati, OH (US); Children's Hospital Medical Center, Cincinnati, OH (US)

(72) Inventors: Xiaoyang Qi, Cincinnati, OH (US); Ying Sun, Cincinnati, OH (US)

(21) Appl. No.: 17/910,105

(22) PCT Filed: Mar. 10, 2021
(86) PCT No.: PCT/US21/21622

§ 371 Date: Sep. 8, 2022

Related U.S. Application Data
(60) Provisional application No. 62/987,662, filed on Mar. 10, 2020.

(51) Int. Cl. C07K 16/18; A61K 9/00; A61K 31/661; A61K 45/06; A61K 31/445; A61K 31/4025; A61P 3/00; A61P 1/16

(52) U.S. Cl. CPC C07K 16/18; C12Y 302/01; A61K 9/0019; A61K 31/661; A61K 45/06; A61K 31/445; A61K 31/4025; A61P 3/00; A61P 1/16

(57) ABSTRACT

A method of treating a subject suffering from Gaucher Disease is provided, the method including administering to the subject an effective amount of a composition including saposin C, dioleoylphosphatidylserine (SapC-DOPS), and acid β-glucosidase (GCase). Also provided is a nanovesicle including saposin C, dioleoylphosphatidylserine, and acid β-glucosidase and pharmaceutical compositions including the SapC-DOPS-GCase nanovesicles.

### CROSS REFERENCE TO RELATED APPLICATION

This application claims priority to U.S. Provisional Application No. 62/987,662 filed Mar. 10, 2020, the contents of which are incorporated herein by reference in their entirety.

### STATEMENT REGARDING FEDERALLY SPONSORED RESEARCH OR DEVELOPMENT

This invention was made with Government support under R21NS095047 awarded by the National Institutes of Health. The Government has certain rights in the invention.

### TECHNICAL FIELD

The present disclosure relates to compositions and methods for the treatment of lysosomal storage diseases. More specifically, the disclosure relates to compositions comprising SapC-DOPS nanovesicles and acid β-glucosidase for the treatment of neuronopathic Gaucher disease.

### BACKGROUND

Gaucher disease (GD) is a common lysosomal storage disease with a frequency of ˜1/57,000 live births. In GD, GBA1 mutations lead to defective acid β-glucosidase (GCase) function and the accumulation of its substrates, glucosylceramide (GluCer) and glucosylsphingosine (GluSph), resulting in multi-organ dysfunction. Typical manifestations of GD type 1 include visceral, hematologic and bone disease reflected by hepatosplenomegaly, anemia, thrombocytopenia, osteopenia, and osteoporosis. In comparison, types 2 and 3 exhibit these visceral signs and also early onset neuronopathic disease, or nGD, with primary brain pathology characterized by neuronal necroptosis and inflammation. Life expectancy is about 1-2 years for GD type 2 patients and up to 30-40 years for those with GD type 3. GBA1 mutations have also been identified as the most common genetic risk factor for Parkinson's and Lewy Body diseases.

Approved therapies for GD include: enzyme replacement therapy (ERT; e.g., imiglucerase, velaglucerase alfa and taliglucerase alfa) and substrate reduction therapy (SRT; miglustat and eliglustat). These therapies are effective on the visceral manifestations of the GD variants, but do not alleviate central nervous system (CNS) symptoms. A new SRT agent (Genz-682452) has shown CNS efficacy in preclinical studies and is in a Phase II clinical trial for GD type 3. Gene therapy using viral vectors has shown promising results, but significant obstacles limit clinical translation. Recent studies using adeno-associated viral (AAV) vector expressing GCase have shown encouraging improvement of CNS disease in GD mouse models, however, immunogenicity and long-term safety of AAV must be established before applying to patients. Additionally, pharmacological chaperones can enhance the mutant GCase stability/trafficking. However, selecting non-inhibitory GCase chaperones for in vivo applications is challenging. Thus, there remains a pressing unmet need for a safe breakthrough approach to nGD treatment.

### SUMMARY

The following summary of the disclosure is provided to facilitate an understanding of some of the innovative features unique to the present disclosure and is not intended to be a full description. A full appreciation of the various aspects of the invention can be gained by taking the entire specification, claims, drawings, and abstract as a whole.

In one embodiment, a method of treating a subject suffering from Gaucher Disease is provided, the method comprising administering to the subject an effective amount of a composition comprising: saposin C and dioleoylphosphatidylserine (SapC-DOPS); and acid β-glucosidase.

In another embodiment, a pharmaceutical composition is provided, the composition comprising: a nanovesicle comprising saposin C, dioleoylphosphatidylserine (SapC-DOPS), and acid β-glucosidase; and a pharmaceutically-acceptable carrier.

In another embodiment, a nanovesicle is provided, the nanovesicle comprising saposin C (SapC), dioleoylphosphatidylserine (DOPS), and acid β-glucosidase.

These and other objects, features, embodiments, and advantages will become apparent to those of ordinary skill in the art from a reading of the following detailed description and the appended claims.

### BRIEF DESCRIPTION OF THE FIGURES

FIG. 1A. SapC-DOPS and SapC activate GCase in a cell-free assay. DOPS as control. (SapC, 0 to 500 nM; DOPS, 0 to 4500 nM). Free GCase incubated with SapC-DOPS or SapC for 30 min before determining the GCase activity.

FIG. 1B. SapC-DOPS increases GCase activity in fibroblasts from Sap C-deficient mice (4L;C* and SapC^{-/-}). Student's t-test. ***, p<0.0001. The cultured cells were incubated with 100 nM SapC-DOPS for 30 min before harvesting for GCase activity assay.

FIG. 1C. K_{d} of GCase interaction with SapC-DOPS compared to saposin B (SapB)-DOPS. The K_{d} values were determined by microscale thermophoresis (MST) using Affinity Analysis v2.1.3 software.

FIG. 1D. K_{d} of SapC interaction with DOPS compared to SapB with DOPS. K_{d}s were determined by microscale thermophoresis (MST) using Affinity Analysis v2.1.3 software.

FIG. 1E. Functional domain of SapC. GCase activity in 4L;C* fibroblasts treated with WT SapC, mutant SapCs or WT SapB formulated with DOPS-GCase. The cells were incubated for 5 days with two changes of fresh medium with the formulation. One-Way ANOVA with Tukey's multiple comparison test. ***, p<0.05. The samples were assayed in triplicates/experiment of 2-3 independent experiments.

FIG. 2A. SapC-DOPS nanovesicles preserve GCase activity of mouse Gba1^{-/-} fibroblasts incubated for 24 or 48 h with SapC-DOPS-GCase or GCase.

FIG. 2B. SapC-DOPS nanovesicles preserve GCase activity of mouse Gba1^{-/-} fibroblasts incubated for 24 or 48 h with SapC-DOPS-GCase or GCase. SapC-DOPS-GCase incubated cells showed higher levels of GCase activity (right panel) and protein (left panel) than GCase incubated cells.

FIG. 2C. GCase activity of human GD type 2 fibroblasts, GM1260 (L444P/P415R) and GM877 (L444P/L444P), incubated with SapC-DOPS-GCase for 24 hours.

FIG. 2D. Human GCase detected in the lysosome (Lamp2a) of Gba1^{-/-} fibroblasts incubated with SapC-DOPS-GCase and GCase for 24 h. The cells with SapC-DOPS-GCase showed higher PCC (Person correlation coefficient) compared to GCase incubated cells (n=8-14 cells), indicating more GCase targeting to lysosomes in SapC-DOPS-GCase incubated cells compared to free GCase incubated cells. Scale bar=100 μm for all images.

FIG. 2E. Effect of mannan on GCase uptake. J774E macrophages were incubated with 80 μg GCase/mL of SapC-DOPS-GCase or free GCase in the presence and absence of Mannan (2 mg/mL). The data presents net GCase activity (increased GCase activity level minus basal/endogenous GCase activity level). The samples were assayed in triplicates/experiment of 2-3 independent experiments.

FIG. 2F. Effect of mannan on GCase uptake. Gba^{-/-} neurons were incubated with 80 μg GCase/mL of SapC-DOPS-GCase or free GCase in the presence and absence of Mannan (2 mg/mL). The data presents net GCase activity (increased GCase activity level minus basal/endogenous GCase activity level). The samples were assayed in triplicates/experiment of 2-3 independent experiments.

FIG. 3A. Tissue distribution of SapC-DOPS-GCase. 4L;C* and WT mice at 38 days of age were i.v. infused with one bolus injection of SapC-DOPS-GCase, vehicle (CP buffer) or free GCase (54.6 mg/kg SapC-DOPS and 0.4 mg/kg GCase; 0.4 mg/kg free GCase). Tissues were collected 3 h post injection or as indicated. GCase protein detected by immunoprecipitation followed with immunoblot in 4L;C* brains with SapC-DOPS-GCase.

FIG. 3B. Tissue distribution of SapC-DOPS-GCase. 4L;C* and WT mice at 38 days of age were i.v. infused with one bolus injection of SapC-DOPS-GCase, vehicle (CP buffer) or free GCase (54.6 mg/kg SapC-DOPS and 0.4 mg/kg GCase; 0.4 mg/kg free GCase). Tissues were collected 3 h post injection or as indicated. GCase protein detected by immunoprecipitation followed with immunoblot in WT brains with SapC-DOPS-GCase.

FIG. 3C. GCase protein was detected in the 4L;C* brains and livers at 1 to 24 hours post injection with SapC-DOPS-GCase.

FIG. 3D. GCase activity increased in SapC-DOPS-GCase treated brain.

FIG. 3E. GCase protein detected in the 4L;C* liver.

FIG. 3F. GCase activity in 4L;C* mouse tissues at 49 days of age treated with SapC-DOPS-GCase, free GCase or vehicle-CP buffer (2 h post i.p. injection of, 2× injection/2 h). SapC-DOPS-GCase was distributed to liver, spleen, lung, lymph nodes and bone marrow cells. SapC-DOPS-GCase-treated mice have significantly higher activity in those tissues than free GCase-treated mice.

FIG. 3G. Distribution of human GCase in brain regions of 4L;C* mice at 38 days of age treated with SapC-DOPS-GCase or free GCase (3 h post one bolus i.v. injection). Human GCase co-stained with VCAM 1 positive endothelium by immunofluorescence (IF) in SapC-DOPS-GCase treated 4L;C* mouse brain. Scale bar is 20 μm.

FIG. 3H. Human GCase detected by IF in the lysosome (Lamp2a) of brain cells. No GCase is detected in free GCase treated 4L;C* brains. Magnifications are 400×.

FIG. 3I. Representative images of GCase protein detected by immunofluorescence in astrocytes (GFAP), neurons (NeuN) and microglia (CD68) in SapC-DOPS-GCase treated 4L;C* mouse brains. Scale bar=20 μm for all images.

FIG. 3J. The graph shows the percentage of brain cells containing GCase.

FIG. 3K. Human GCase positive cells (stars) determined by IF were distributed in 4L;C* mouse brain regions that show inflammation stained by anti-CD68 antibody. 4L;C* mice were i.v. infused with SapC-DOPS-GCase or free GCase. Hematoxylin stains nuclei. Scale bar is 3 mm. n=3 mice, replicated assays of 2-3 independent experiments, n=>5 images/mouse. Student's t-test. *, p<0.05; **, p<0.01; ***, p<0.001.

FIG. 4A. Therapeutic efficacy of Sap-DOPS-GCase. 4L;C* mice were treated with SapC-DOPS-GCase and vehicle or free GCase by daily i.p. injections of SapC-DOPS-GCase from day 21 to 27 followed by tail i.v. vein injection from day 28 to terminal age, 3 times per week. Hindlimb clasping was significantly improved in SapC-DOPS-GCase treated 4L;C* mice compared to saline treated 4L;C* mice. 4L;WT mice is the normal control. ANOVA test. **, p<0.01; ***, p<0.001.

FIG. 4B. Sap-DOPS-GCase treatment significantly improved right and left strides of 4L;C* mice at 50 and 55 days of age compared to vehicle (CP buffer) or saline control. Student's t-test. ***, p<0.001.

FIG. 4C. Compared to free GCase, vehicle and untreated 4L;C* mice, SapC-DOPS-GCase treatment significantly prolonged survival of 4L;C* mice. Log-Rank (Mantel-Cox) test (p<0.05).

FIG. 4D. GCase substrate, GluCer was significantly reduced in treated 4L;C* brain (n=3, 50 days of age). Student's t-test (p<0.05).

FIG. 4E. Treated 4L;C* brain regions (cortex, thalamus, brain stem and midbrain) showed significantly reduced inflammation determined by anti-GFAP (astrocyte) staining (n=3, 50 days of age). The signal densities of GFAP, were quantified with Image J software and presented as a bar graph. Scale bar is the same for all images. n=>5 images/mouse. Student's t-test. ***, p<0.001.

FIG. 5A. SapC-DOPS in vitro and in vivo efficacy. SapC-DOPS-CVM detected in the thalamus region of 4L;C* mouse brain that has inflammation stained positive by anti-CD68 antibody. SapC-DOPS-CVM also showed in control 4L;WT mice brain that has no inflammation. Magnification of images are 100×(left panel). Bar graph (right panel) shows CVM levels in 4L;C* and control mice brain in thalamus region. 4L;C* mice at 40 days of age were i.v. injected with 200 μL SapC-DOPS-CVM (SapC=0.4 mg/mL, DOPS=0.082 mg/mL, CVM=0.04 μM). Tissues were collected 24 h post injection.

FIG. 5B. 4L;C* mice were administered with SapC-DOPS, SapC-DOPS-GCase or vehicle (CP buffer) by daily i.p. injections of SapC-DOPS or SapC-DOPS-GCase from day 21 to 27, followed by tail i.v. vein injection of SapC-DOPS or SapC-DOPS-GCase, 3 times per week, from day 28 to terminal age. (b) Increased GCase activity in the brain of 4L;C* mice treated with SapC-DOPS (n=3, 40 days of age). Student's t-test. **, p<0.01.

FIG. 5C. SapC-DOPS treated 4L;C* mice showed significantly prolonged survival compared to vehicle-4L;C*. The survival is longer in SapC-DOPS-GCase-treated 4L;C* compared to SapC-DOPS-treated 4L;C* mice. Log-Rank (Mantel-Cox) test (p<0.05).

FIG. 5D. In comparison to SapC-DOPS, SapC-DOPS-GCase treated 4L;C* mice showed significant improved left stride and right stride at 45 days of age (n=6). Student's t-test. *, p<0.05.

FIG. 6A. Phosphatidylserine (PS)-mediated inflamed brain targeting determined by PS-binding protein, lactadherin. Lactadherin blocked SapC-DOPS-CVM targeting into 4L;C* mouse brains. Significantly diminished CVM signals were observed in 4L;C* brains injected with lactadherin (Lact) compared with BSA of whole brain and sagittal brain cuts. Non-symptomatic, non-injected littermate, 4L/WT, brains, used as controls, have no CVM signal.

FIG. 6B. Relative CVM signal levels are shown in the graph for 4L*C brains (left panel) and brain cuts (right panel). Lact was administered by i.v. to 4L;C* mice (Lact, 20 μg/mouse, n=5), BSA (20 μg/mouse, n=5) or PBS (100 μL, n=2) 30 min prior to SapC-DOPS-CVM (200 μL/mouse, i.v.). The brains were perfused and fixed in 4% PFA. CVM signals were acquired by ex-vivo IVIS imaging. Ex=640 nm, Em=700 nm, Exposure=0.5 sec. Student's t-test.

FIG. 6C. Lact distributes in neural cells and inflamed brain regions. L;C* and 4L/WT control mice were injected with 100 or 200 mL of Lact (83 mg/mL) by i.v. The brain sections were stained with anti-lactadherin-FITC in combination with anti-CD68 (microglia), anti-Tuj1 (Neuron) and anti-VCAM1 (endothelium). Lact signals were around microglia and neuron cells, and on endothelium.

FIG. 6D. Signal levels (RFU, relative fluorescence unit) of Lact in inflamed 4L;C* brain regions. Student's t-test (n=4 mice). *, p<0.05; **, p<0.01; ***, p<0.001; ****, p<0.0001.

FIG. 7A. Formulation scheme of SapC-DOPS-GCase and diagram illustration of SapC-DOPS-GCase. Formulated SapC-DOPS-GCase was subjected to ultracentrifugation. GCase activity and protein were determined in supernatant (free form) and pellet (bound form).

FIG. 7B. Immunoblots of GCase protein quantitation in bound and free form in formulation optimization. (b) Optimization of GCase in formulation. SapC (0.1 mM) and DOPS (0.9 mM) was formulated with various GCase (0.7, 1.0, 1.3 μM). The control free GCase is 0.3 μM. Bound GCase of 1.3 μM G Case in the formulation had highest activity and protein (Table 1, FIG. 15).

FIG. 7C. Optimization of DOPS in the formulation. Fixed SapC (0.1 mM) vs. DOPS (0.5-1.1 mM) formulated with 1.3 μM GCase. The ratio of SapC to DOPS at 0.1 to 0.7-0.9 mM is optimal for SapC-DOPS-GCase formulation (Table 2, FIG. 16).

FIG. 8A. Stability of SapC-DOPS-GCase in serum and medium. GCase activity of SapC-DOPS-GCase and free GCase in mouse serum (pH7.4) and medium (10% DMEM, pH 7.2) were measured from 0 to 8 hours at 37° C. Stability presented as EC₅₀ values (hrs) calculated by PRISM software.

FIG. 8B. Stability curves in serum. Data is from duplicate assays of 3 replicates each.

FIG. 8C. Stability curves in media. Data is from duplicate assays of 3 replicates each.

FIG. 9. The pixel scatter diagrams of GCase-green (GFP 488 nm) vs. Lamp2a-red (Texas Red 595 nm). Human GCase detected in the lysosome (Lamp2a) of Gba1^{-/-} fibroblasts incubated with SapC-DOPS-GCase and GCase for 24 hrs. The pixel scatter diagrams of the cells in FIG. 2D showed the Pearson correlation coefficient (PPC) analysis of co-expression (r) of GCase and Lamp2a signals in lysosome. SapC-DOPS-GCase treated cells (r=0.771) had more GCase in lysosome than GCase treated cell (r=0.333). The untreated cells had no GCase in lysosomes (r=0.00003). A representative image of a total 8-14 cells from each group.

FIG. 10. SapC and GCase are co-localized in brain cells. Human GCase was co-stained with human SapC in brain cells and blood vessel by immunofluorescence in SapC-DOPS-GCase treated 4L;C* mouse brain. Scale bar is 20 μm. Fluorescence signals were acquired with a Zeiss Apotome 200M. Pearson correlation coefficient (PPC) analysis showed co-expression (r) of GCase and SapC in brain cells (r=0.7069±0.0384 in brain cells, mean±SEM, n=13) distributed in thalamus, midbrain and cortex (not shown), and in blood vessels (arrows). Free GCase injected 4L;C* brain had no SapC or GCase signals. Representative images from each group. 4L;C* mice at 38 days of age treated with SapC-DOPS-GCase or free GCase (54.6 mg/kg SapC-DOPS and 0.4 mg/kg GCase; 0.4 mg/kg free GCase), 3 hours post one bolus i.v. injection. n=2 mice. 4% PFA fixed brain sections were stained with mouse monoclonal antibody to human SapC (1:100 of 1 mg/mL generated in the Qi's Lab) followed by goat anti-mouse-488 nm, and rabbit anti-human GCase (1:200) followed by goat anti-rabbit-Texas Red. The samples were mounted with Anti-fade mounting medium with DAPI.

FIG. 11A. SapC-DOPS-GCase treatment reduced brain inflammation and neurodegeneration. Brain inflammation was determined by anti-CD68 (microglia) antibodies in the brains of SapC-DOPS-GCase treated 4L;C* mice. Treated 4L;C* brain regions (cortex, thalamus, midbrain, and brainstem) showed significantly reduced inflammation.

FIG. 11B. Neurodegeneration in 4L;C* mouse brains was determined by FJC staining. Treated 4L;C* brain regions (thalamus, midbrain, and brain stem) showed significantly reduced FJC signals compared to those in untreated 4L;C* mice, indicating reduced neurodegeneration (left panel). 4L;C* mice were treated with SapC-DOPS-GCase with the group 2 regime. Brains were collected at 50 days of age. The signal densities of CD68 and FJC were quantified with Image J software and presented as a bar graph (right panel). Scale bar is the same for all images. Student's t-test. *, p<0.05; **, p<0.01; ***, p<0.001. n=3 mice.

FIG. 12. Lactadherin blocks cell surface PS-targeting of SapC-DOPS nanovesicles in vitro. SapC-DOPS-CVM binds to brain tumor Gli36 cells (1st to 3rd columns from left). Pretreatment of SapC-DOPS-CVM with free lactadherin did not reduce the PS-binding effect of SapC-DOPS-CVM on cells (2nd columns from left). Cells pre-incubated with lactadherin showed reduced SapC-DOPS-CVM binding (3rd column form left). The controls (DOPS-CVM with and without lactadherin, 4th and 5th columns from left) do not show sufficient binding to the cells. All cell samples were measured for CVM fluorescence by flow cytometry. Student's t-test (p<0.05).

FIG. 13A. Lymphatic pathway is involved in SapC-based nanovesicles targeting to CNS. SapC-DOPS-CVM was not detected in lectin positive blood vessels in mouse brains that were i.v. administered lectin along with either SapC-DOPS-CVM (top panel) or saline (bottom panel).

FIG. 13B. Positive GCase signal is co-localized with LYVE1 (lymphatic vessel marker) in SapC-DOPS-GCase treated 4L;C* mouse brain meninges (top panel). GCase alone (middle panel) and LYV1 alone (bottom panel) are shown.

FIG. 13C. SapC-DOPS-CVM was detected in lymph nodes 24 hours post i.v. injection of SapC-DOPS-CVM. DAPI stains nucleus. SapC-DOPS-CVM (top panel) and saline (bottom panel) are shown.

FIG. 13D. WT and K14-VEGFR3-Ig (lack brain microlymphatics) mice that were engrafted with orthotropic brain tumor LLC-GFP cells (top panel, graph and image) were i.v. injected with SapC-DOPS-CVM at 12 days of age. CVM signal was detected in the WT mouse brain tumors, but was undetectable in K14-VEGFR3-Ig mouse brain tumors or WT mice without tumors 18 hours post injection (bottom panel, graph and image).

FIG. 14A. Proposed mechanisms are illustrated for systemic GCase delivery by SapC-DOPS nanovesicles into lysosomes of inflamed brain cells. SapC-DOPS-GCase nanovesicles cross the inflamed blood vessels and are released into the brain interstitium via: (1) PS-mediated transcytosis. SapC binds surface phosphatidylserine (PS) on the endothelial cells and leads to internalization of the nanovesicles followed by transportation across the cell, and (2) EPR/vesicle diffusion. SapC-DOPS-GCase migrates through the compromised vessel gaps between endothelial cells by EPR or vesicle diffusion. Subsequently, the SapC-DOPS-GCase targets inflamed brain cells and is transported to lysosomes by (3) PS-mediated uptake similar to (1), and (4) receptor-mediated uptake through specific binding of GCase and the mannose receptor.

FIG. 14B. Proposed mechanism of SapC-based nanovesicles targeting to CNS. Data indicate that meningeal-lymphatics are required for SapC-based vesicle uptake in diseased brains and strongly support that SapC-based nanovesicles are taken up by the inflamed-brain of GD mice by a PS-facilitated pathway and via a meninges-mediated CNS-lymphatic loop.

FIG. 15. Table showing optimization of GCase levels in formulation (Table 1, top panel) and optimization of DOPS levels in the formulation with GCase (Table 2, bottom panel).

FIG. 16. Table 3, showing interaction of GCase with SapC-DOPS.

### SEQUENCE LISTING

Applicant hereby incorporates by reference a CRF sequence listing submitted herewith having a file name Sequence_Listing_10738_848.txt (6 KB) created on Mar. 7, 2021.

The nucleic and/or amino acid sequences listed in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. In the accompanying sequence listing:

SEQ ID NO: 1 represents a peptide sequence of saposin C (SapC); and

SEQ ID NO: 2 represents a peptide sequence of human acid β-glucosidase (GCase).

### DETAILED DESCRIPTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all terms (including technical and scientific terms) used herein have the same meaning as commonly understood by one of ordinary skill in the art to which this disclosure belongs. It will be further understood that terms such as those defined in commonly used dictionaries should be interpreted as having a meaning that is consistent with their meaning in the context of the relevant art and the present disclosure, and will not be interpreted in an idealized or overly formal sense unless expressly so defined herein.

The terms “treat,” “treatment,” and “treating,” as used herein, refer to a method of alleviating or abrogating a disease, disorder, and/or symptoms thereof in a subject.

As used herein a “subject” or “patient” refers to a mammal. Optionally, a subject or patient is a human or non-human primate. Optionally, a subject or patient is a dog, cat, horse, sheep, cow, rabbit, pig, or mouse.

An “effective amount” is defined herein in relation to the treatment of Gaucher disease as an amount of SapC-DOPS-GCase that will decrease, reduce, inhibit, or otherwise abrogate the progression of Gaucher disease. In specific embodiments, the effective amount is an amount that will decrease, reduce, inhibit, or otherwise abrogate neurodegeneration associated with neuronopathic Gaucher disease. In some embodiments, the therapeutic agent(s) can be delivered regionally to a particular affected region or regions of the subject's body. In some embodiments, wherein such treatment is considered more suitable, the therapeutic agent(s) can be administered systemically. For example, the compound can be administered parenterally. In a specific embodiment, a therapeutic agent is delivered intravenously.

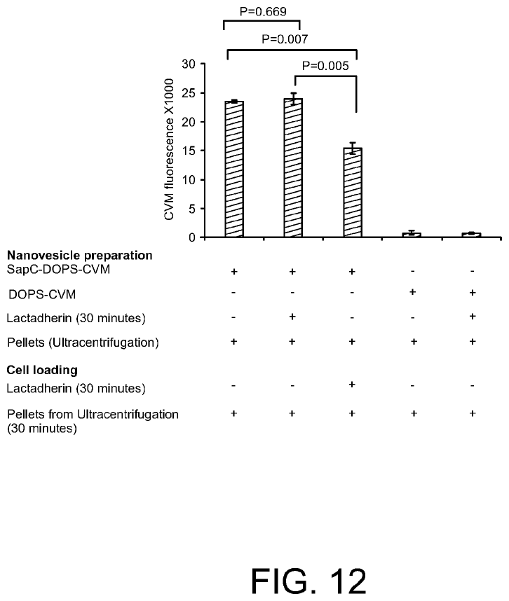

SapC-DOPS refers to a stable nanovesicle composed of saposin C (SapC), a lysosomal protein that catabolizes glycosphingolipids, and the phospholipid dioleoylphosphatidylserine (DOPS) (FIG. 12). SapC-DOPS is further described in U.S. Pat. No. 8,937,156, issued Jan. 20, 2015, which is incorporated herein by reference. In one embodiment, SapC has a protein sequence consisting of SDVYCEVCEFLVKEVTKLIDNNKTEKEILDAFDKMCSKLPKSLSEECQEVVDT YGSSILSILLEEVSPELVCSMLHLCSG (SEQ ID NO. 1). In some embodiments, SapC comprises a protein sequence having at least 75%, at least 80%, at least 85%, at least 90%, at least 95%, at least 98%, or at least 99% sequence identity to SEQ ID NO: 1.

SapC is a small lysosomal glycoprotein contained with three other saposins (A, B and D) in a single precursor, prosaposin, which are present in all normal tissues. SapC functions as a critical optimizer of GCase activity, protects GCase from protease degradation, and prevents GCase inhibition by α-synuclein. Mutations in the SapC codons of the human or mouse prosaposin genes, PSAP or Psap, respectively, lead to SapC deficiency and GD/nGD-like diseases. SapC deficiency-GD-like diseases have a much lower frequency than GBA1-based GD. A novel complex, SapC-DOPS (SapC coupled with the phospholipid, dioleoylphosphatidylserine; DOPS), selectively targets and effectively delivers SapC to extra-cranial inflammatory sites and, by crossing the compromised blood brain barrier (BBB), to primary brain tumors and brain metastases of breast and lung cancers. Thus, SapC-DOPS nanovesicles are capable of enhancing mutant GCase function and also provide a biological vehicle for delivering GCase into the central nervous system (CNS).

SapC-DOPS and modifications, variants, analogs, and formulations are known in the art. U.S. Pat. No. 7,834,147 describes many such compositions and formulations, which are useful in the present disclosure. U.S. Pat. No. 7,834,147 is hereby incorporated by reference.

Acid β-glucosidase, or GCase, is an enzyme that acts on glucosylceramide (also called glucocerebroside). When the GCase enzyme is defective, as in Gaucher disease, glucosylceramide and its deacylated lysolipid, glucosylsphingosine, accumulate and cause multi-organ dysfunction. Various recombinant human GCase formulations are known in the art, including but not limited to, imiglucerase (Genzyme), velaglucerase alfa (Shire), and taliglucerase alfa (Pfizer). In one embodiment, GCase comprises a protein sequence consisting of MEFSSPSREECPKPLSRVSIMAGSLTGLLLLQAVSWASGARPCIPKSFGYSSVV CVCNATYCDSFDPPTFPALGTFSRYESTRSGRRMELSMGPIQANHTGTGLLLT LQPEQKFQKVKGFGGAMTDAAALNILALSPPAQNLLLKSYFSEEGIGYNIIRV PMASCDFSIRTYTYADTPDDFQLHNFSLPEEDTKLKIPLIH-RALQLAQRPVSLL ASPWTSPTWLKTNGAVNGKGSLKGQPGDIYHQTWARYFVKFLDAYAEHKL QFWAVTAENEPSAGLLSGYPFQCLGFTPEHQRDFIARDLGPTLANSTTHHN VR LLMLDDQRLLLPHWAKVVLTDPEAAKYVHGIAVHWYLDFLAPAKATLGET HRLFPNTMLFASEACVGSKFWEQSVRLGSWDRGMQYSHSIITNLLYHVVGW TDWNLALNPEGGPNWVRNFVDSPIIVDITKDTFYKQPMFYHLGHFSKFIPEGS QRVGLVASQKNDLDAVALMHPDGSAVVVVLNRSSKDVPLTIKDPAVGFLETI SPGYSIHTYLWRRQ (SEQ ID NO: 2). In some embodiments, GCase comprises a protein sequence having at least 75%, at least 80%, at least 85%, at least 90%, at least 95%, at least 98%, or at least 99% sequence identity to SEQ ID NO: 2.

“Nanovesicle,” as used herein, refers to stable lipid vesicles having a size range of from about 10 to about 800 nm, or more specifically from about 150 to about 300 nm. In embodiments, the nanovesicles described herein are present in physiological buffers and may comprise a range of protein-to-lipid ratios. In a specific embodiment, GCase interacts with SapC-DOPS nanovesicles with a binding constant, Kd, of about 29 nM.

The term “pharmaceutically-acceptable excipient,” as used herein, means any physiologically inert, pharmacologically inactive material known to one skilled in the art, which is compatible with the physical and chemical characteristics of the particular active agent selected for use. Pharmaceutically acceptable excipients include, but are not limited to, polymers, resins, plasticizers, fillers, lubricants, diluents, binders, disintegrants, solvents, co-solvents, buffer systems, surfactants, preservatives, sweetening agents, flavoring agents, pharmaceutical grade dyes or pigments, and viscosity agents. In specific embodiments, the pharmaceutically-acceptable excipients are excipients suitable for intravenous injection formulations.

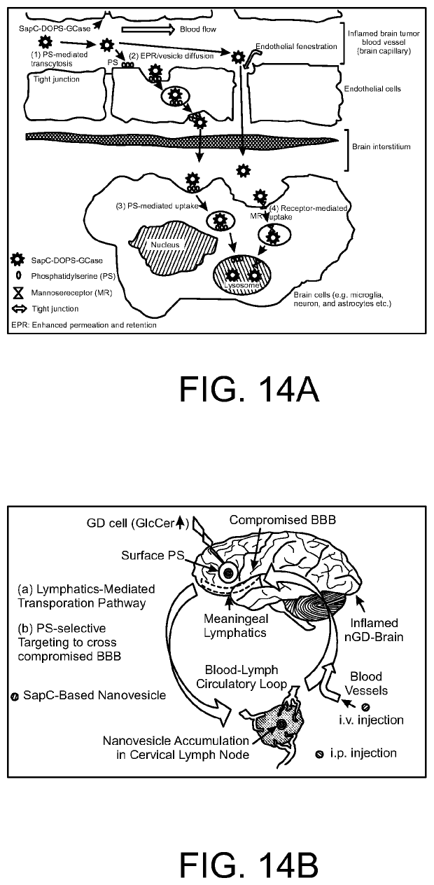

There is a major unmet medical need for a GD treatment with direct effects on the CNS. Herein, we developed a novel strategy using BBB-penetrating SapC-DOPS nanovesicles through a non-invasive, intravenous administration, to transport functional GCase into the nGD brain which resulted in therapeutic effects. Formulating GCase into SapC-DOPS preserved GCase function and enhanced stability and uptake into cells, primarily via mannose receptor-independent pathways. We also demonstrate that SapC-DOPS-GCase crosses the BBB of normal and inflamed brains via a mechanism that requires PS-targeting (see FIGS. 14A, 14B). In vivo efficacy of SapC-DOPS-GCase in a mouse model of nGD showed significant improvement in CNS inflammation, neurodegeneration, survival and neurological phenotype even when the treatment started around onset of disease signs, demonstrating the therapeutic value of SapC-DOPS-GCase in lessening the nGD.

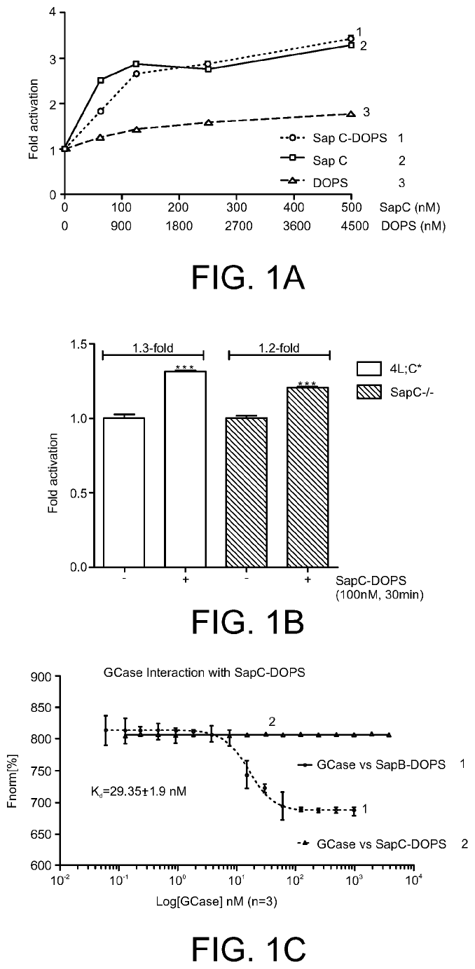

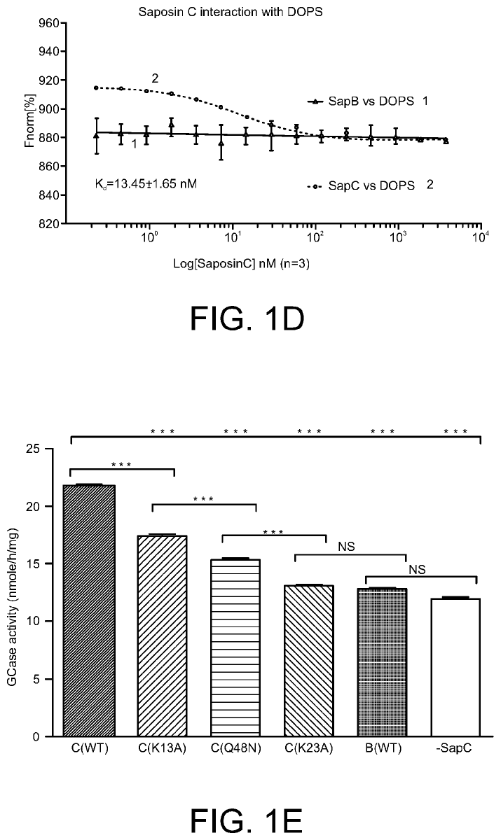

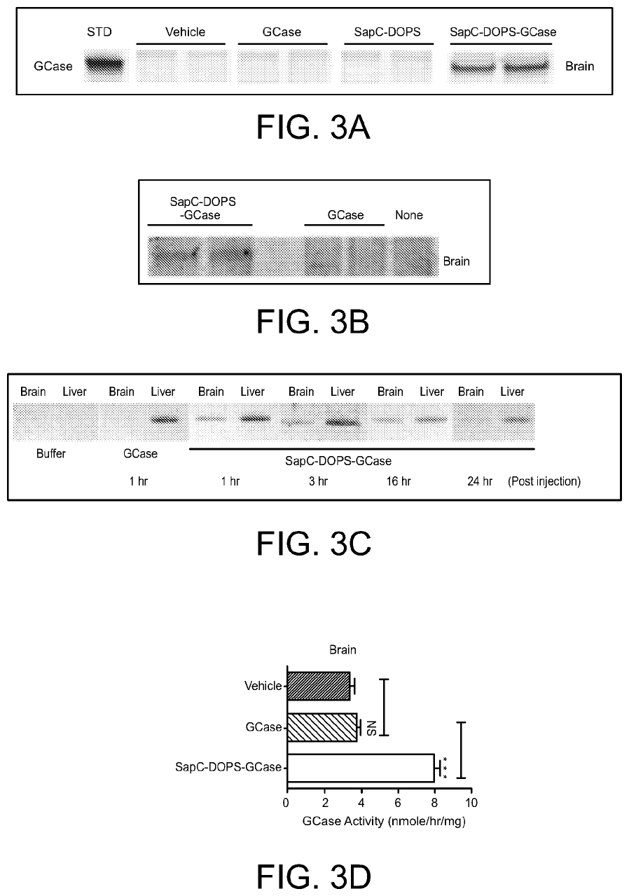

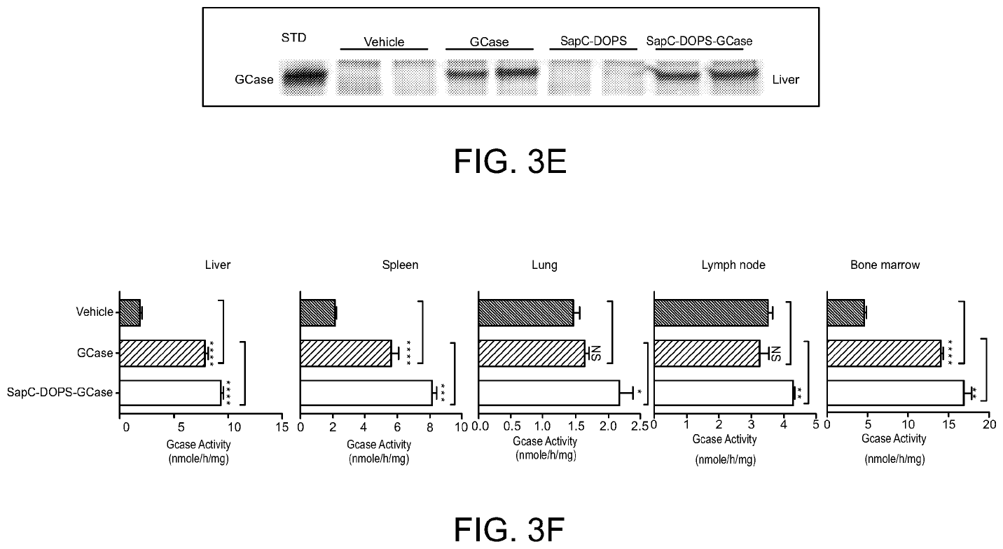

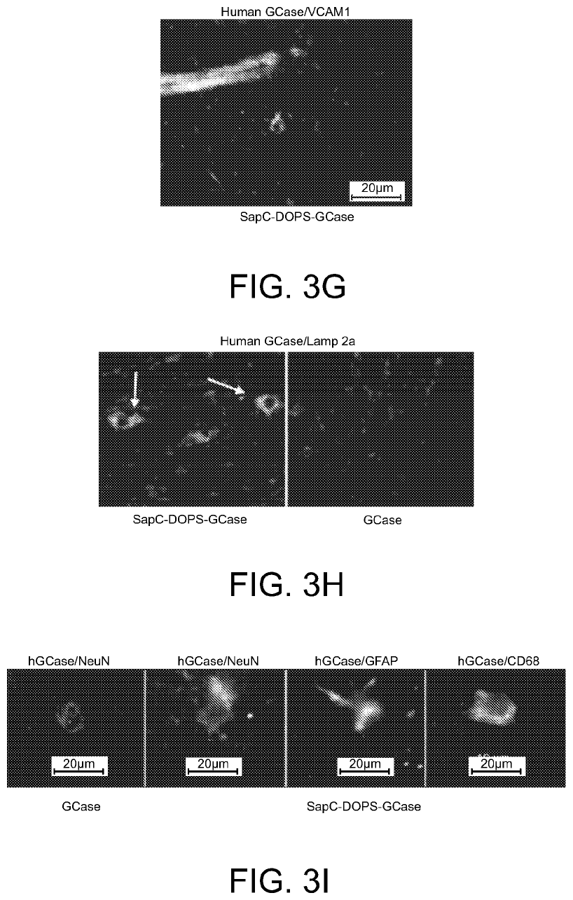

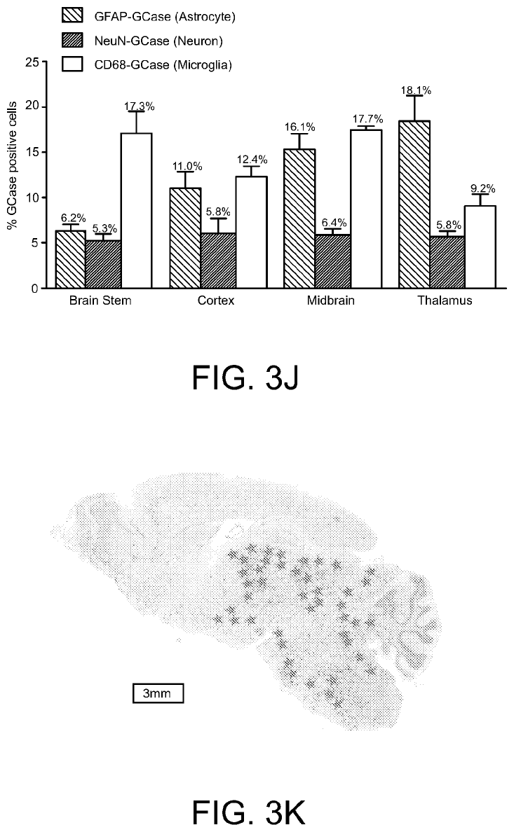

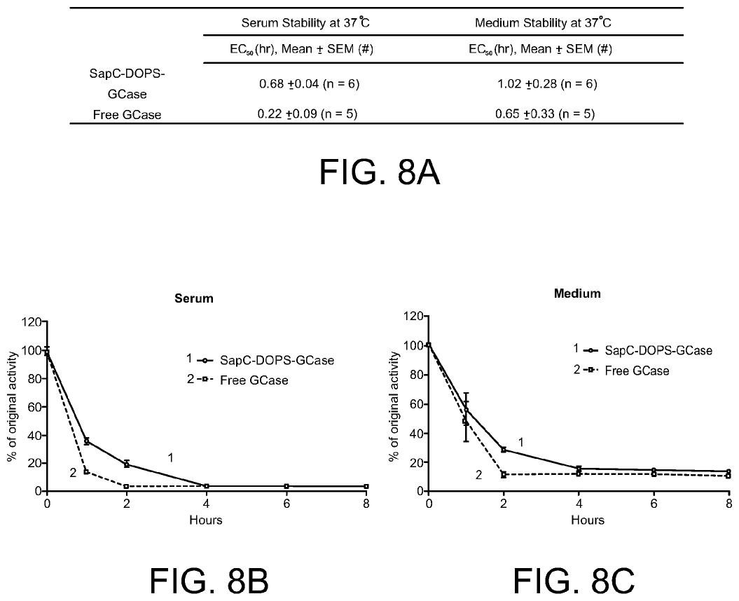

In this study, GCase binding to SapC-DOPS revealed a K_{d} of 29 nM indicating a tight interaction of GCase with SapC-DOPS nanovesicles. In the SapC-DOPS-GCase formulation, GCase is sheltered by the lipid bilayer that allows slow release of GCase and protects it from rapid denaturation by the neutral pH of plasma or culture media (FIGS. 1 and 8A-8C), and delays clearance into the reticuloendothelial system (FIG. 3F). EC₅₀ of GCase in this formulation is three times more than free GCase in serum (FIG. 8A-8C). Thus, SapC-DOPS-GCase provides a more stable ERT formulation than current ERT agents.

GCase therapeutics are mannose-terminated recombinantly-produced human enzyme and its cell uptake is preferential for the mannose receptor. Reduced mannose receptor-dependency of SapC-DOPS-GCase suggests that the GCase has surface and interior membrane occupancy of the SapC-DOPS nanovesicles. Thus, significant amounts of GCase are delivered through fusion of SapC-based vesicles with the cell and organelle membranes. Consequently, SapC-DOPS-GCase can access a wider variety of cell types allowing the enzyme into the organs that are inaccessible by ERT, such as lymph nodes, lung and brain (FIGS. 3A-3K). While ERT has no effect on lymph nodes in GD type 3 patients, SapC-DOPS-GCase accesses lymph nodes (FIG. 3F) and may provide an option for GD patients with massive mesenteric or other lymphadenopathy.

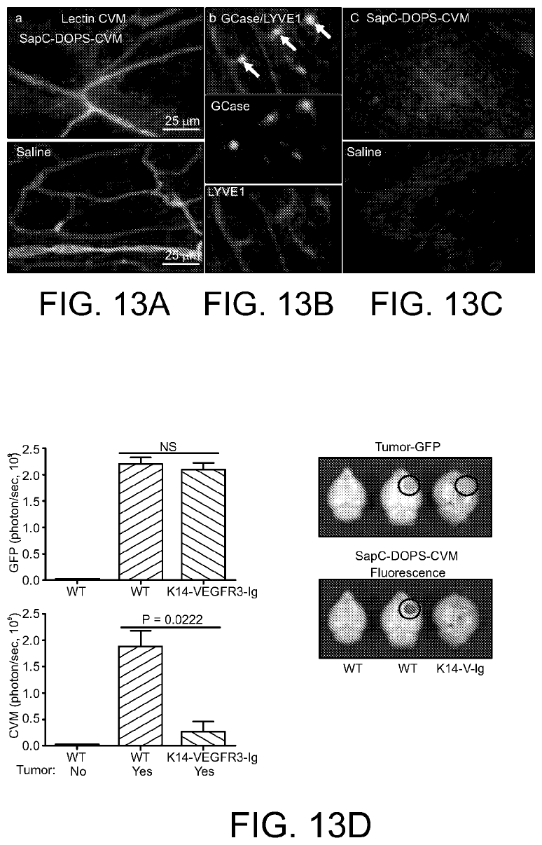

Phosphatidylserine (PS) is normally found on the inner, ‘invisible’ leaflet of the plasma membrane. SapC, a membrane-associated protein, has a favorable binding affinity with unsaturated, negatively charged PS. Multiple lines of evidence indicate that inflamed and damaged cells (i.e., apoptotic or necrotic cells) often have abnormal surface PS. PS-selective SapC-based nanovesicles are taken up by the brain, especially by PS-exposed enriched inflamed-brains, by crossing compromised BBB in several neuronal disease mouse models including brain tumor, multiple sclerosis, and epilepsy. As SapC-DOPS targeting is blocked by pre-treatment with the PS-specific binding protein, Lact-C2, such nanovesicles must directly interact and fuse with the PS-exposed cell membranes and cell surface PS as a major, specific target for SapC-DOPS-GCase (FIG. 13A). PS is a general surface lipid biomarker of inflammatory cells that is suitable for targeted therapy and diagnosis using SapC-based nanovesicles as well as other PS-selective agents.

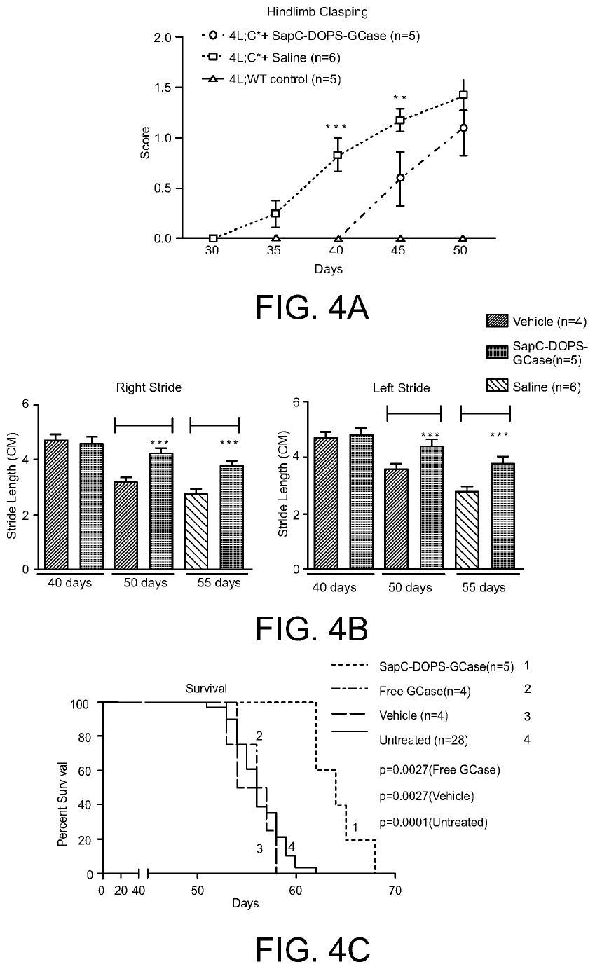

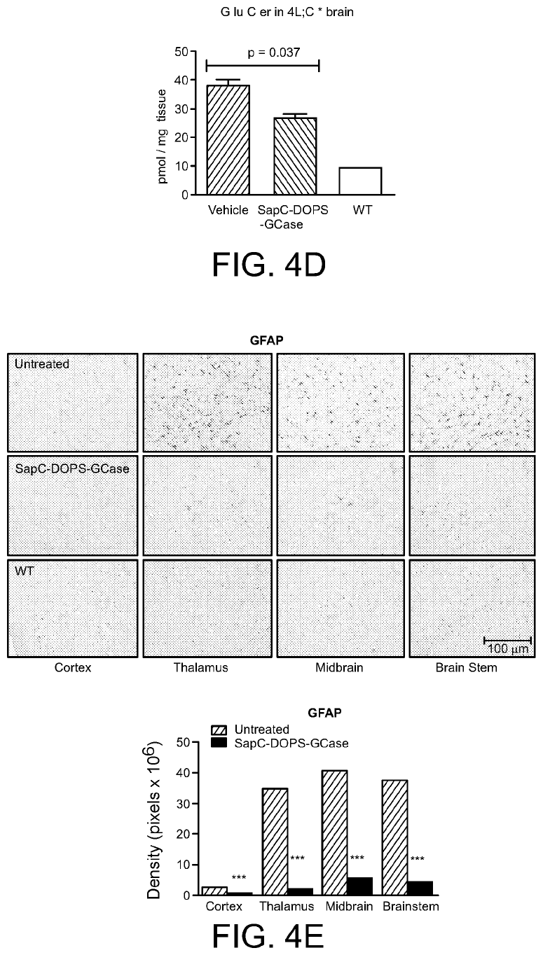

Nanoscale lipid-based vesicles are taken up by the lymphatic system. Indeed, SapC-DOPS nanovesicles or SapC-DOPS-GCase significantly accumulated in mouse spleen, lymph nodes, and liver (FIGS. 3 and 13A-13D). In prior studies, we have also demonstrated that the nanovesicle was associated with macrophages and neutrophils in inflammatory arthritic joints. Meningeal lymphatics are a reservoir in the CNS blood-lymph circulatory loop. Here, the long term accumulation of the systemically administrated fluorescent SapC-DOPS vesicles in meningeal tissues (FIGS. 13A and 13B) was shown and this signal was significantly reduced in the animals with a defective meningeal lymphatic system (FIG. 13D). In addition, the SapC-DOPS-GCase nanovesicles are co-localized with inflammatory cells (microglia/macrophage) from brain (FIGS. 4A-4E). These findings suggest that SapC-DOPS-GCase may be co-transported by inflammatory cells from the spleen and/or lymph nodes into nGD brains via a compromised blood-lymph circulatory loop (FIG. 14B).

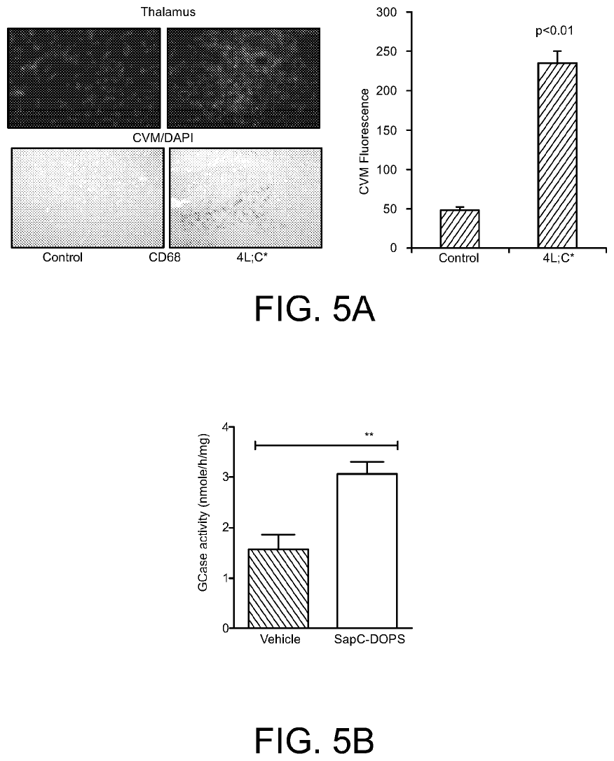

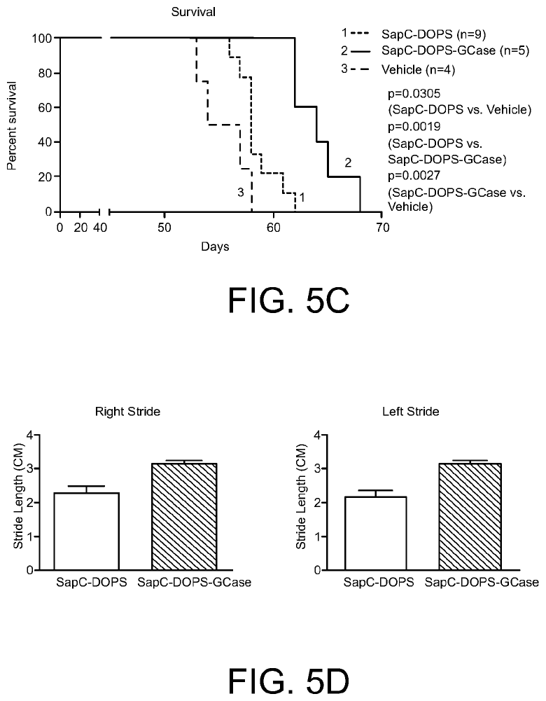

SapC activates GCase to achieve maximal enzymatic function both in vitro and in vivo and, importantly can induce some GCase mutants to achieve near normal GCase activity. This enhanced activity of mutant GCase stimulated by SapC-DOPS may be therapeutic in selected GD patients who have specific “susceptible” mutations. The GCase activation domain of SapC is localized in the carboxyl-terminal region and the primary physiological function of SapC has been identified from lysosomal storage diseases caused by deficiencies of SapC in humans and mice. Seven cases of a GD-like disease linked to mutations of SapC have been reported in Europe and China. Therefore, therapeutic SapC-DOPS may benefit GD due to defective GCase as well as nGD with SapC deficiency. The data showed that SapC-DOPS enhances GCase activity ex vivo and in vivo (FIGS. 5A-5D). Notably, SapC-DOPS treatment improved survival and gait abnormalities in 4L;C* mice even when the treatment was started at the age when disease signs appears, demonstrating its CNS efficacy, but SapC-DOPS-GCase showed better effects.

Although ERT is the current standard of care for GD, the available ERT agents do not access particular organs, e.g. brain, lung alveoli, and lymph nodes, and are unable to treat the associated inflammation in such organs. Additionally, available ERT agents have no direct effects on nGD since the BBB blocks CNS access to the enzyme. The BBB-penetrating SapC-based nanovesicles developed here delivered exogenous functional GCase into the CNS and establish a potential brain-specific enzymatic therapy for nGD. SapC-DOPS-GCase allows intravenous administration of enzyme treatment for the CNS disease, which has advantages over more invasive procedures, e.g. intracerebral and intrathecal delivery of biologicals into the CNS. SapC-DOPS-GCase provides more benefits than conventional ERT by stabilizing GCase and delivering enzyme into more organs, e.g. brain, lung and lymph nodes that are inaccessible by conventional ERT. These studies have shown that functional GCase transported by SapC-DOPS nanovesicles is taken up by the inflamed CNS but also by normal brain, which allows SapC-DOPS-GCase treatment at the stages with and without inflammatory breach of BBB. The delivered GCase distributes to the affected regions and reduces substrate accumulation in nGD, subsequently mitigating the phenotype and pathology. While disease improvement was achieved when the treatment started around onset of disease signs, there is a possibility that treatment initiation at a younger age, e.g., postnatal 3 days in this model, could significantly improve the efficacy. The basis for the selective CNS regional uptake of SapC-DOPS-GCase in the 4L;C* mice requires additional investigations. The positive outcomes from these preclinical evaluations in the mouse model of nGD strongly support the translational potential of SapC-DOPS-GCase for GD treatment.

The present disclosure supports SapC-DOPS-GCase as a novel therapeutic approach for GD therapy and potentially other lysosomal storage diseases or neurodegenerative disorders. SapC-DOPS-GCase is more stable than conventional ERT and accessible to both visceral and CNS organs, providing a potentially first ERT for managing both CNS and visceral symptoms. This study demonstrates a new mechanism of CNS targeting of SapC-DOPS via a PS-mediated and lymphatic circulation system. SapC-DOPS is a GMP-grade biological and has exhibited a superb safety profile in a Phase 1 clinical trial (Clinical-Trials.gov Identifier: NCT02859857). The ERT of velaglucerase alfa used in this study is a FDA approved drug for GD treatment. nGD will remain lethal without improvements in treatment but since both of the biological entities have good safety profiles, SapC-DOPS-GCase has the potential to rapidly translate to improved patient care.

### Nanovesicles and Pharmaceutical Formulations

In one embodiment, a nanovesicle is provided, the nanovesicle comprising saposin C (SapC), dioleoylphosphatidylserine (DOPS), and acid β-glucosidase. In embodiments, the nanovesicle comprises SapC and DOPS in a molar ratio of about 1:7. In embodiments, the SapC-DOPS nanovesicles comprise a phospholipid bilayer comprising DOPS molecules, wherein SapC protein molecules are associated with the bilayer on the outer surface of the nanovesicle, the interior of the nanovesicle, or both the outer surface of the nanovesicle and the interior of the nanovesicle. In embodiments, SapC-DOPS nanovesicle size ranges from about 150 to about 300 nm.

In embodiments, the acid β-glucosidase comprises a protein sequence consisting of SEQ ID NO: 2. In another embodiment, the acid β-glucosidase comprises a protein sequence having at least 75%, at least 80%, at least 85%, at least 90%, at least 95%, at least 98%, or at least 99% sequence identity to SEQ ID NO: 2. In embodiments, the acid β-glucosidase comprises imiglucerase, velaglucerase alfa, taliglucerase alfa, and combinations thereof.

In embodiments, acid β-glucosidase is bound to SapC in the presence of DOPS in the nanovesicle via non-covalent interactions. In embodiments, the noncovalent interaction between acid β-glucosidase and SapC is selected from the group consisting of hydrophobic interactions, van der Waals forces, and combinations thereof.

In another embodiment, a pharmaceutical composition is provided, the composition comprising: a nanovesicle comprising saposin C, dioleoylphosphatidylserine (SapC-DOPS), and acid β-glucosidase; and a pharmaceutically-acceptable carrier. Pharmaceutical formulations can be prepared for intravenous or parenteral administration, as discussed in detail below.

The effective dosage of SapC-DOPS-GCase in animal disease models, the use of which is within the scope of embodiments described herein, will vary somewhat from subject to subject and will depend upon the condition of the subject and the route of delivery. As a general proposition, a dosage of from about SapC (24 mg/kg), DOPS (12.4 mg/kg), and GCase (0.27 mg/kg) to about SapC (48 mg/kg), DOPS (24.8 mg/kg), and GCase (0.8 mg/kg) will have efficacy. In one embodiment, a specific dose for IV injection includes SapC (36 mg/kg), DOPS (18.6 mg/kg), and GCase (0.40 mg/kg), with all weights being calculated based upon the weight of the active compound.

In embodiments, the SapC-DOPS-GCase composition formulation comprises from about 0.05 to about 0.2 mM SapC; from about 0.4 to about 1.2 mM DOPS; and from about 1.0 to about 2.6 μM GCase.

In embodiments, the SapC-DOPS-GCase composition formulation comprises about 0.05, 0.06, 0.07, 0.08, 0.09, 0.1, 0.11, 0.12, 0.13, 0.14, 0.15, 0.16, 0.17, 0.18, 0.19, or 0.2 mM SapC; about 0.4, 0.5, 0.6, 0.7, 0.8, 0.9, 1.0, 1.1, or 1.2 mM DOPS; and about 1.0, 1.1, 1.2, 1.3, 1.4, 1.5, 1.6, 1.7, 1.8, 1.9, 2.0, 2.1, 2.2, 2.3, 2.4, 2.5, or 2.6 μM GCase.

In a specific embodiment, the proportions of SapC, DOPS, and GCase in the SapC-DOPS-GCase nanovesicle composition formulation include about 0.1 mM SapC about 0.7 to about 0.9 mM DOPS:about 1.3 μM GCase.

In accordance with the presently disclosed methods, SapC-DOPS-GCase can be administered intramuscularly, subcutaneously, intra-arterially, or intravenously as a solution, suspension, or emulsion. In a specific embodiment, SapC-DOPS-GCase is formulated for intravenous administration.

In addition to the SapC-DOPS-GCase, the pharmaceutical compositions can contain other additives, such as pH-adjusting additives. In particular, useful pH-adjusting agents include acids, such as hydrochloric acid, bases or buffers, such as citric phosphate, sodium lactate, sodium acetate, sodium phosphate, sodium citrate, sodium borate, or sodium gluconate. In a specific embodiment, the pharmaceutical composition comprises citric phosphate buffer.

In embodiments, the pH of the pharmaceutical composition is selected to preserve maximum GCase activity. In certain embodiments, the pH is an acidic pH of from about 3.8 to about 6.8. In a specific embodiment, the pharmaceutical composition comprises a pH of about 5.6.

Further, the compositions can contain antimicrobial preservatives. Useful antimicrobial preservatives include methylparaben, propylparaben, and benzyl alcohol. The antimicrobial preservative is typically employed when the formulation is placed in a vial designed for multi-dose use.

### Methods of Treating Gaucher Disease

SapC-DOPS-GCase nanovesicles and compositions including the nanovesicles are useful for treating lysosomal storage disorders, including Gaucher disease. In a specific embodiment, the Gaucher disease comprises neuronopathic Gaucher disease (nGD). In another specific embodiment, the Gaucher disease comprises Gaucher disease type 1, type 2, and type 3. In a very specific embodiment, the Gaucher disease is selected from the group consisting of Gaucher disease type 2 and Gaucher disease type 3.

In some embodiments, the methods of treating Gaucher disease in a subject in need thereof comprise administering to the subject an effective amount of a composition comprising: saposin C and dioleoylphosphatidylserine (SapC-DOPS); and acid β-glucosidase (SapC-DOPS-GCase nanovesicles). In embodiments, the SapC-DOPS-GCase is present in a pharmaceutical formulation as described above. In specific embodiments, the SapC-DOPS-GCase is administered intravenously. In another specific embodiment, the SapC-DOPS-GCase crosses the blood brain barrier (BBB) of the subject.

In addition, it will be appreciated that the therapeutic benefits for the treatment of Gaucher disease can be realized by combining treatment with one or more additional therapeutic agents or treatments effective for the treatment of Gaucher disease. The choice of such combinations will depend on various factors including, but not limited to, the type of Gaucher disease, the age and general health of the subject, the aggressiveness of disease progression, and the ability of the subject to tolerate the agents that comprise the combination.

In embodiments, the second therapeutic agent is selected from an enzyme replacement therapy, a substrate reduction therapy, a pharmacological chaperone, and combinations thereof. In a specific embodiment, the second therapeutic agent is an enzyme replacement therapy selected from the group consisting of imiglucerase, velaglucerase alfa, taliglucerase alfa, and combinations thereof. In another specific embodiment, the second therapeutic agent is a substrate reduction therapy selected from the group consisting of miglustat, eliglustat, venglustat, and combinations thereof. In another specific embodiment, the second therapeutic agent is a pharmacological chaperone. Suitable pharmacological chaperones are disclosed, for example, in Zimran, A., et al., Pilot study using ambroxol as a pharmacological chaperone in type 1 Gaucher disease, Blood Cells Mol Dis 50: 134-37 (2013); and Kumar, D. T et al., Chapter 8: A comparative computational approach toward pharmacological chaperones (NN-DNJ and ambroxol) on N370S and L44P mutations causing Gaucher's disease, Advances in Protein Chemistry and Structural Biology, vol. 114: 315-39 (2019); which references are incorporated by reference in its entirety. In a specific embodiment, the pharmacological chaperone is selected from the group consisting of ambroxol hydrochloride, N-(n-nonyl)deoxynojirimycin (NN-DNJ), and combinations thereof.

Combination treatments involving SapC-DOPS-GCase and another therapeutic agent, can be achieved by using both agents at substantially the same time, i.e., concurrently. Alternatively, treatment with the compound of the present invention can precede or follow treatment with the other agent by intervals ranging from minutes to weeks, i.e., sequentially.

Embodiments can be described with reference to the following numbered clauses, with preferred features laid out in dependent clauses.

1. A method of treating a subject suffering from Gaucher Disease, the method comprising administering to the subject an effective amount of a composition comprising: saposin C (SapC) and dioleoylphosphatidylserine (SapC-DOPS); and acid β-glucosidase (GCase).
2. The method according to clause 1, wherein the composition comprises SapC-DOPS-GCase nanovesicles.
3. The method according to any of the preceding clauses, wherein a molar ratio of saposin C (SapC) to dioleoylphosphatidylserine (DOPS) in the SapC-DOPS-GCase nanovesicles is about 1:7.
4. The method according to any of the preceding clauses, wherein the GCase is bound to SapC in the presence of DOPS via non-covalent interactions selected from the group consisting of hydrophobic interactions, van der Waals forces, and combinations thereof.
5. The method according to any of the preceding clauses, wherein SapC, DOPS, and GCase are present in the composition in a ratio of about 0.1 mM SapC:about 0.7 mM to about 0.9 mM DOPS:about 1.3 μM GCase.
6. The method according to any of the preceding clauses, wherein the composition is administered intravenously.
7. The method according to any of the preceding clauses, wherein the composition crosses a blood brain barrier of the subject.
8. The method according to any of the preceding clauses, wherein Gaucher Disease comprises neuronopathic Gaucher Disease (nGD).
9. The method according to clause 8, wherein the nGD is selected from the group consisting of Gaucher Disease type 2 and Gaucher Disease type 3.
10. The method according to any of the preceding clauses, further comprising administering to the subject a second therapeutic agent effective for the treatment of Gaucher Disease.
11. The method according to clause 10, wherein the second therapeutic agent is selected from an enzyme replacement therapy, a substrate reduction therapy, and a pharmacological chaperone.
12. The method according to clause 11, wherein the second therapeutic agent is an enzyme replacement therapy selected from the group consisting of imiglucerase, velaglucerase alfa, taliglucerase alfa, and combinations thereof.
13. The method according to clause 11, wherein the second therapeutic agent is a substrate reduction therapy selected from the group consisting of miglustat, eliglustat, venglustat, and combinations thereof.
14. The method according to clause 11, wherein the second therapeutic agent is a pharmacological chaperone selected from the group consisting of ambroxol hydrochloride, N-(n-nonyl)deoxynojirimycin (NN-DNJ), and combinations thereof.
15. A pharmaceutical composition comprising: a nanovesicle comprising saposin C (SapC), dioleoylphosphatidylserine (SapC-DOPS), and acid β-glucosidase (GCase); and a pharmaceutically-acceptable carrier.
16. The pharmaceutical composition according to clause 15, wherein saposin C (SapC) and dioleoylphosphatidylserine (DOPS) are present in a molar ratio of about 1:7.
17. The pharmaceutical composition according to any of clauses 15-16, wherein SapC, DOPS, and GCase are present in the composition in a ratio of about 0.1 mM SapC:about 0.7 mM to about 0.9 mM DOPS:about 1.3 μM GCase.
18. The pharmaceutical composition according to any of clauses 15-17, wherein the GCase is bound to SapC in the presence of DOPS via non-covalent interactions selected from the group consisting of hydrophobic interactions, van der Waals forces, and combinations thereof.
19. The pharmaceutical composition according to any of clauses 15-18, formulated for intravenous administration.
20. A nanovesicle comprising saposin C (SapC), dioleoylphosphatidylserine (DOPS), and acid β-glucosidase (GCase).
21. The nanovesicle according to clause 20, wherein a molar ratio of SapC to DOPS is about 1:7.
22. The nanovesicle according to any of clauses 20-21, wherein the GCase is bound to SapC in the presence of DOPS via non-covalent interactions selected from the group consisting of hydrophobic interactions, van der Waals forces, and combinations thereof.

### EXAMPLES

### Example 1: Materials

The following reagents were from commercial sources: conduritol-B-epoxide (CBE), neurobasal, FJC staining kit (Fluoro-Jade® C), mouse anti-NeuN monoclonal antibody (MAB377), rat anti-b actin, -Lamp1 and -Lamp2a antibodies (EMD Millipore, Bedford, Mass.); mammalian protein extraction reagent (M-PER; Invitrogen), DMEM, RPMI medium and Pierce BCA protein assay kit (ThermoFisher Sci., Waltham, Mass.); rat anti-mouse LYVE1 monoclonal antibody (ALY7; eBioscience, Waltham, Mass.); mouse anti-GFAP monoclonal antibody (Clone2E1.E9) and mouse anti-Tuj1 monoclonal antibody (StemCell, Cambridge, Mass.); mouse anti-CD68 monoclonal antibody (Biolegend, San Diego, Calif.); mouse anti-VACM1 (MR106, Ebioscience, San Diago, Calif.); goat anti-rabbit FITC (A-32731), goat anti-mouse FITC (A-21131), goat anti-rabbit Texas Red (A-11037), goat anti-mouse Texas Red (A-32742) (Invitrogen, Carlsbad, Calif.); NuPAGE Gels (3-8%, 4-12%, 10%) and Bis-Tris Buffer, sodium acetate buffer, SeaBlue Plus2 protein standard, PVDF membrane, iBlot transfer kit, and iBind Cards (Life Technologies. Carlsbad, Calif.); AP color reagent and AP conjugate substrate kit (Bio-Red, Hercules, Calif.); VECTASHIELD mounting medium containing DAPI (Vector, Burlingame, Calif.); annexin V-FITC, Dynabeads® protein G kit and BS3 (Invitrogen, CA); lactadherin and lactadherin-FITC (Haematologic Technologies, Essex Junction, Vt.); mannan (Sigma, St. Louis, Mo.).

SapC prepared under Good Manufacturing Practices (GMP) and HPLC purified is produced using the pET expression system in E. coli cells by the Changji Bio-Tech Company (Changzhou, China). The endotoxin level in the SapC preparations was <0.1 EU/mg, achieving a safety level for clinical use. GCase is Velaglucerase alfa with mannosyl-terminated chains that is provided by Shire. The nanovesicle size is measured at ˜190-200 nm in dimension by photon correlation spectroscopy utilizing a N4 plus particle size analyzer and TEM imaging showed uniform SapC-DOPS nanovesicles.

### Example 2: SapC-DOPS-GCase Formulation

SapC and DOPS was mixed at molar ratio 1:7 in 250 mL 0.1 M Citrate Phosphate (CP) buffer (pH 5.6) with 80 mg/mL (1.33 μM) GCase (1 U=10 mg of Velaglucerase alfa, GCase). The formulation mixture was bath sonicated for 15 mins at 4° C. and then diluted to 1 mL with CP buffer.

### Example 3. Experimental Methods

### 1. Binding Assay for SapC and DOPS and GCase and SapC-DOPS Nanovesicles

Binding was determined by measuring Kd using a Microscale Thermophoresis (MST) assay system on a Monolith NT.115. MicroScale NanoTemper. Temperature related intensity change (TRIC) together with thermophoresis contribution to the variation of fluorescence intensity was determined. NBD-PS [2-(4-nitro-2,1,3-benzoxadiazol-7-yl)aminoethyl-phophatidylserine (Avanti Polar Lipids Inc., Alabaster, Ala.) was used to fluorescently label DOPS. DOPS in chloroform was mixed with NBD-PS in DMSO at the molar ratio of 3:1. The organic solvent was evaporated under nitrogen gas. Upon suspension in CP buffer (0.1 M Citrate Phosphate buffer, pH 5.6), the mixture solutions were bath sonicated for 30 mins at 4° C. This preparation was tested with SapC or SapB to measure the K_{d} for binding to DOPS. To make fluorescently labeled compounds, SapC-DOPS, SapB-DOPS or SapC-DOPC (1,2-dioleoyl-sn-glycero-3-phosphocholine), each at 1:7 molar ratio was formulated with NBD-PS at 3 to 1 molar ratio, followed by sonication at 4° C. Those mixtures were used to measure the K_{d} values of GCase with SapC-DOPS, SapB-DOPS and SapC-DOPC. For the non-labeled interacting competitors: purified GCase was prepared from 3124 nM to 0.096 nM in CP buffer by 2-fold serial dilutions into 16-point concentrations in a 96-well plate. These serially diluted GCase were mixed with a fixed concentration of 15,000 nM labeled SapC-DOPS-NBDat ratio of 1:1.

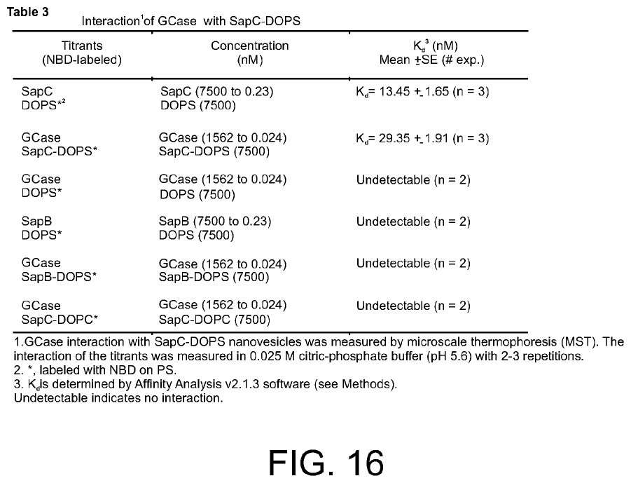

The final concentration of the reaction mixtures ranged from GCase/SapC-DOPS-NBD of 1562 nM/7500 nM for the 1st point to 0.024 nM/7500 nM for the 16th point (FIG. 16, Table 3). In the measurement of SapC or SapB with DOPSNBD for K_{d} interaction, SapC or SapB were serially diluted (0.46 to 15000 nM, 16 points) and then freshly mixed with labeled DOPS-NBD at a fixed concentration of 15000 nM with a 1:1 ratio. The final concentration of SapC was 0.23-7500 nM and DOPS-NBD was 7500 nM in the reaction mixture. All samples were prepared immediately prior to use. The labeled and unlabeled materials at each concentration were mixed prior to the start of the measurement. The thermophoresis time was 30 secs with dual reading setting (LED: 20%/40%; MST: 40%/50%). All experiments were repeated 2 to 3 times on different days. The raw data were analyzed to determine K_{d} values and plotted using MO Affinity Analysis v2.1.3 software (NanoTemper, München, Germany).

### 2. Estimation for the Number of SapC and GCase Molecules in Each DOPS Nanoparticle

Number of molecules in each DOPS nanoparticle is based on the simple geometry and average size measurements for DOPS liposomes (Nₜₒₜ=4π[r²+(r−h)²]/a, r=95 nm, h=5 nm, a=0.65 nm²). Using this equation, the estimated DOPS liposome nanoparticle contains 3.31×10⁵ DOPS molecules (Nₜₒₜ). Total number of DOPS liposomes is 6.55×10⁹ per mL calculated by the equation: Nₗᵢₚₒ=(M_{lipid}×NA)/(Nₜₒₜ×1000). Nₗᵢₚₒ is liposome numbers. M_{lipid} is lipid molar concentration. NA is Avogadro constant. The number of SapC molecules per DOPS nanovesicles is calculated based on the 0.4 μM SapC and 3.6 μM DOPS in the formulation. The number of SapC molecules per DOPS nanovesicles is estimated based on 0.625 μM GCase with 75% bound on SapC-DOPS.

### 3. GCase Activity Assay

Two assay buffer systems were used, a Tc/Tx system (1% Na-taurocholate/1% Triton X-100) for measuring GCase activity in cell free assay, serum, cells and visceral tissues (FIGS. 1, 2, 3F, 8A-8C, and 15). The brain PS (BPS) system (0.5 mM BPS) is used to determine SapC's effect on brain GCase activity by homogenizing the brains in 1×PBS and incubated in 0.5 mM BPS (FIGS. 3D and 5A). The GCase activity assay was carried out as previously described. Liou B, et al., Analyses of variant acid beta-glucosidases: effects of Gaucher disease mutations, J Biol Chem 281(7):4242-53 (2006). Protein concentrations of cells and tissues were determined by the BCA assay using bovine serum albumin (BSA) as a standard. In the cell-free assays, the dilution of GCase (Velaglucerase alfa, 1 mg/mL) was 1:2000. The final concentration of polysorbate 20 was ˜ 4 nM which has no impact on GCase activity.

### 4. Cell Culture

Fibroblasts from Gba1 null (Gba1^{-/-}), 4L;C* and SapC deficient (C^{-/-}) mice and human GD type 2 patient fibroblasts (GM1260: GBA1 L444P/P415R and GM877: GBA1 L444P/L444P, Coriell Institute, Camden, N.J.) were cultured in DMEM with 10% FBS. Transformed Gba1^{-/-} neurons, kindly provided by Dr. Ellen Sidransky at NIH, were maintained in Neurobasal Medium. Macrophage mannose receptor positive, J774E macrophage cell lines were maintained with 10% FCS/DMEM as described (Du, H., et al., The role of mannosylated enzyme and the mannose receptor in enzyme replacement therapy, Am J Hum Genet 77: 1061-1074 (2005)). For GCase uptake experiments, equal units of GCase (8 U/mL) as SapC-DOPS-GCase or free as GCase freshly prepared in CP buffer (pH 5.6) were added to the cells (1×10⁶ cells/well) and incubated for 24 or 48 hours. GCase activity and protein concentration were determined (Xu, Y. H., et al. Dependence of reversibility and progression of mouse neuronopathic Gaucher disease on acid beta-glucosidase residual activity levels, Mol Genet Metab 94: 190-203 (2008)). The cells cultured on chamber slides were processed for lysosomal localization of GCase by immunofluorescence (IF) using Lamp2a as a lysosomal marker. For mannose receptor-mediated GCase uptake experiments: J774E and Gba1^{-/-} neuron cells were treated with a mannose receptor inhibitor, Mannan (2 mg/mL) overnight. The cells were collected and washed with glycine (0.9%)/saline to remove the surface attached GCase prior to GCase activity measurement.

### 5. SapC-DOPS-GCase Serum/Medium Stability

Freshly formulated SapC-DOPS-GCase in CP buffer (pH 5.6) was tested for its stability in serum (pH 7.4) from wild type (WT) mice and DMEM medium (pH 7.2, 10% FCS) over 8 h. GCase activities were measured at 0, 1, 2, 4, 6 and 8 h with the Tc/Tx system. The same amount of free GCase was set as a control to compare its activity versus SapC-DOPS-GCase at same condition. The assays were repeated twice, each in triplicates. EC₅₀ was calculated using GraphPad Prism 7.

### 6. Immunoprecipitation (IP) of GCase

IP was performed using Dynabeads® protein G kit to quantify human GCase protein in the mice. Protein G was prepared by crosslinking the trapping antibody, rabbit anti-human GCase using bis(sulfosuccinimidyl) substrate (BS3, Thermo-Fisher). Mouse brain or liver lysates (1 mg protein from nGD mice and 10 mg protein from WT mice) were mixed with 80 mL of protein G beads cross-linked with anti-GCase antibody and incubated at 4° C. overnight. GCase was eluted from the beads using glycine, pH ˜7.2-8 and analyzed by immunoblot. Following elution, the beads were denatured with SDS buffer and showed no detectable GCase.

### 7. Immunoblotting

The cells and tissues were homogenized in Mammalian-Protein Extraction Reagent (M-PER) and subjected to electrophoresis on 4-12% NuPAGE gels. 3-actin was used as the loading control. The proteins were transferred to PVDF membranes using an iBlot 2 gel transfer device (Life Technologies) following the manufacturer's instructions. The blots were incubated with rabbit anti-human GCase antibody (1/1000) or anti-3-actin (1:5000) antibody overnight at 4° C. in 1.5% BSA/1.5% milk/PBS buffer. Pure GCase (Velaglucerase alfa) was used as a standard (10, 20 and 40 ng protein) and loaded on the same gel with the samples for quantitation of GCase protein. The IP GCase was analyzed using goat anti-human GCase antibody. The signals were detected with AP Conjugate Substrate Kit according to manufacturer's instructions. Optical densities of protein bands on the immunoblots were quantified by Image J 1.51j 4 (NIH, Bethesda, Md.).

### 8. GD Mouse Model and Treatment

4L;C* mice harbor V394L/V394L Gba1 (4 L) and saposin C^{-/-} (C*) homozygosity. The 4L;C* mice were originally generated in the background of C57BL/6J/129SvEV. To minimize mixed background interference with behavioral testing, a C57BL/6J strain of 4L;C* mice was generated. Here, 4L;C* mice in the C57BL/6J background were generated by first crossing of V394L/V394L Gba1 and saposin C^{-/-} in C57BL/6J/129SvEV with WT C57BL/6J mice for 10 generations, and then back-crossing of C57BL/6J V394L/V394L Gba1 and C57BL/6J saposin C^{-/-}. The C57BL/6J 4L;C* mice developed the same neurological phenotype as C57BL/6J/129SvEV 4L;C* mice and with an average life span of ˜56 days. These mice have a sufficient lifespan to allow for the current studies of biochemical correction of GCase deficiency by SapC-DOPS-GCase and to assess phenotype improvement by these treatments.

The 4L;C* mice were treated with SapC-DOPS-GCase, free GCase or vehicle at 10 μl/g body weight. Starting day 21, when the tail vein was not accessible, the formulation was administered by daily intraperitoneal (i.p.) injections at dose of total SapC-DOPS (109.2 mg/kg) with GCase (0.8 mg/kg). Tail vein of intravenous (i.v.) injection started at day 28, 3 times per week, at a dose of total SapC-DOPS (54.6 mg/kg) and GCase (0.4 mg/kg). The parallel control mice were either not injected, injected with vehicle (CP buffer or saline), free GCase in CP buffer (i.p—0.8 mg/kg, i.v.—0.4 mg/kg) or SapC-DOPS (i.p—109.2 mg/kg, i.v.—54.6 mg/kg). In some experiments, the mice were administered with acute i.p. or i.v. dosing as indicated in the figure legend. Mouse body weights were recorded daily. The mice were monitored for survival and assessed for phenotype development during the treatment and used for analysis of GCase activity, protein and substrate, and brain pathology. The non-4L;C* littermates (4L;WT and 4L;C+/−) do not show abnormal behavior or pathology and have a normal life span. The strain and age-matched WT mice and non-4L;C* littermates were used as controls. We did not observe gender differences in preliminary studies; age and strain matched mice of both genders were included in all the analyses. All mice were housed under pathogen-free conditions in the animal facility according to IACUC approved protocol (2018-0056) at Cincinnati Children's Hospital Research Foundation.

### 9. CVM in K14-VEGFR3-lg

The K14-VEGFR3-Ig mouse is a brain microlymphatic mouse model. K14-VEGFR3-Ig mice were obtained from Drs. Kari Alitola (University of Helsinki, Finland) and Melody Swartz (Institute for Molecular Engineering, University of Chicago, Chicago, Ill.). WT and K14-VEGFR3-Ig mice with engrafted orthotropic brain tumors (100,000 LLC-GFP cells) were treated with 250 μL SapC-DOPS-CVM (SapC=0.4 mg/ml, DOPS=0.082 mg/ml, CVM (CellVue Maroon)=0.04 μM) via the tail vein at 12 days of age. The brains of SapC-DOPS-CVM administered mice were collected 24 hours post i.v. injection. CVM signal was observed by IVIS imaging (Ex=640 nm; Em=700 nm, 0.1 sec) as described (Qi, X. et al., Cancer-selective targeting and cytotoxicity by liposomal-coupled lysosomal saposin C protein, Clinical cancer research: an official journal of the American Association for Cancer Research 15: 5840-51, (2009)).

### 10. Glycosphingolipid Analysis

GluCer and GluSph in cells and tissues were analyzed at the Medical University of South Carolina Lipidomics Shared Resource: Analytical Unit. The concentration of GluCer and GluSph in the tissues was normalized to mg tissue weight, and in the cells, was normalized by mg protein of the cell lysate as described previously. Sun, Y. et al., Substrate compositional variation with tissue region and Gba1 mutations in mouse models—implications for Gaucher disease, PloS one 8: (2013).

### 11. Immunohistochemistry

Brain tissues were collected after transcardial perfusion with saline and fixed in cold 4% paraformaldehyde (PFA) for processing frozen blocks. CD68 monoclonal antibody staining was as described (Sun, Y., et al., Neuronopathic Gaucher disease in the mouse: viable combined selective saposin C deficiency and mutant glucocerebrosidase (V394L) mice with glucosylsphingosine and glucosylceramide accumulation and progressive neurological deficits, Hum Mol Genet 19: 1088-97 (2010)). The BenchMark XT IC/ISH Staining Module (Ventana Medical System, Tucson, Ariz.) was used for immunohistochemistry studies of CD68 and GFAP at CCHMC Pathology Research Core. Tissue sections were counterstained with Hematoxylin. CD68 and GFAP signals were quantified using Fiji for Image J.

### 12. Fluoro-Jade® C (FJC) Staining

FJC (AG325, Millipore, MA) is a polyanionic fluorescein derivative which selectively binds to degenerative neurons for evaluation of neurodegeneration. Frozen brain sections were air-dried and dipped in 80% ethanol/1% sodium hydroxide, 70% ethanol, and 0.06% potassium permanganate for 5, 2, and 10 min, respectively. The sections were rinsed with distilled water and then incubated with 0.0004% FJC in 0.1% acetic acid for 20 min. FJC staining was detected under a fluorescent microscope at 480 nm excitation and 525 nm emission. Images were acquired through a 20×objective with a Zeiss Apotome 200M, and the fluorescence of FJC-positive cell signals was quantified using Fiji for Image J.

### 13. Immunofluorescence

The mouse tissues and the cells on chamber slides were fixed with 4% PFA, permeabilized with 0.3% Triton X100 in PBS, and quenched with 0.05M NH₄Cl. The cells were blocked with 1.5% BSA and 1.5% non-fat milk in PBS at room temperature for 1 hr. Frozen PFA fixed tissue sections were treated with 0.3% Triton X100 in PBS and blocked with 1.5% BSA and 10% goat serum in PBS. Primary antibodies were applied to the cells or tissue sections and incubated overnight at 4° C. Dilutions of primary antibodies were as follows: rabbit anti-human GCase antibody (1:200); rat anti-Lamp2a or rat anti-Lamp1 antibodies (1:250); rat anti-mouse LYVE1 monoclonal antibody (1:250); mouse anti-GFAP monoclonal antibody (1:250); mouse anti-NeuN monoclonal antibody (1:500); and mouse anti-CD68 monoclonal antibody (1:250). Following washing with PBS plus 0.05% Tween-20 (10 min, 3 times), the samples were incubated for 1 h at room temperature with secondary antibodies:goat anti-rabbit-FITC (1:500), goat anti-rat or anti-mouse-TX Red (1:500). The samples were washed with PBS plus 0.05% Tween-20 (10 min, 3 times) and mounted with Anti-fade mounting medium with DAPI. Fluorescence signals were acquired with a Zeiss Apotome 200M. Signals from the images were quantified by Fiji for Image J.

### 14. Behavioral Testing

Sensorimotor function was assessed in the mice by gait analyses. Each mouse's hindpaws were brushed with non-toxic paint. As mice walked into their home-cage through an alley they left their hindpaw prints on the paper underneath. Stride length and base width were determined by measuring the distance between hindpaw prints. The treated 4L;C* and vehicle 4L;C* mice, and non-4L;C* littermates (normal phenotype), were tested for gait at each time point.

Hindlimb clasping was tested by tail suspension. The mouse was lifted away from all surrounding objects by grasping tail at the base. Hindlimb position was monitored for 30 seconds and scored. Score 0 is when hindlimbs are consistently splayed outward, away from the abdomen. Score 1 is when one hindlimb is retracted toward the abdomen for >50% of 30 seconds. Score 2 is when both hindlimbs are partially retracted toward the abdomen for >50% of 30 seconds. Score 3 is when hindlimbs are entirely retracted and touching the abdomen for >50% of 30 seconds. The mice were tested with two trials at each time point, 10 mins apart between trials.

### 15. Lactadherin Administration and CVM Quantitation

4L;C* mice at 42-43 days of age were i.v. injected with 100 μL lactadherin (20 μg/mouse), BSA (20 μg/mouse) or PBS. Thirty minutes later, each mouse was given SapC-DOPS-CVM (200 μL/mouse) by i.v. injection. Mice were euthanized 3 hours after the SapC-DOPS-CVM injection and perfused with saline followed by 4% PFA. Brains and meninges were collected for ex-vivo IVIS imaging and post-fixed in 4% PFA for 24 hrs followed by 30% sucrose for histological studies. CVM signal was observed by IVIS imaging (Ex=640 nm; λEm=700 nm; Exposure=0.5 s) as described (Blanco, V. M., et al., Phosphatidylserine-selective targeting and anticancer effects of SapC-DOPS nanovesicles on brain tumors, Oncotarget 5: 7105-18 (2014)).

### 16. Distribution of Administered Lactadherin in Brain Cells

Age, strain and sex matched 4L;C* mice and heteroallelic (4L/WT) control mice were injected i.v. with 100 and 200 μL bovine lactadherin-FITC (83 μg/mL in PBS). The brains were collected 3 hours post-injection, transcardiac perfused and fixed in 4% PFA. The brain sections were stained with rat-anti-mouse CD68 monoclonal antibody (MCA1957, Bio-Red, 1:200) or anti-Tuj1 with secondary antibody conjugated with Texas Red (Invitrogen, 1:500), respectively. Images of brain sections were acquired with a Nikon C-plus2 microscope and quantification of fluorescence signals on these images were analyzed by ImageJ.

### 17. Statistical Analysis

The data were analyzed by Student's t-test or ANOVA test, and Log-Rank test for survival using GraphPad Prism 6.

### Example 4. Characterization of SapC-DOPS-GCase Formulation

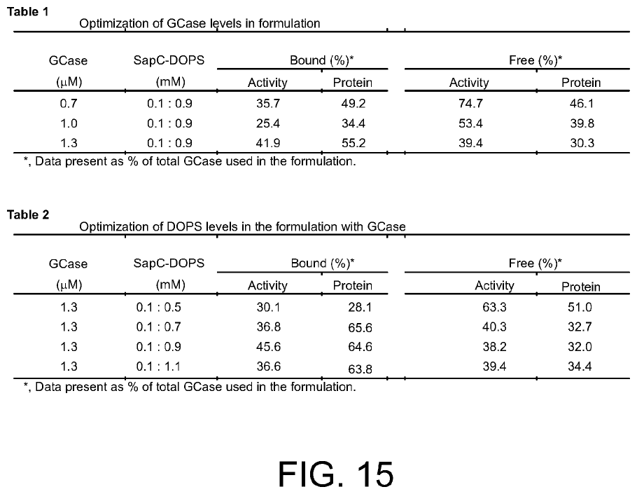

A strong GCase and SapC interaction has been demonstrated in the presence of unsaturated, and acidic phospholipids. Microscale thermophoresis (MST) experiments were conducted to determine the interaction/binding of GCase with SapC-DOPS. MST detects changes in the hydration shell, charge or size of molecules by measuring changes of the mobility of molecules in microscopic temperature gradients. Using fluorescent NBD-phosphatidylserine (PS) as probe in DOPS, GCase showed interaction with SapC-DOPS with a binding constant K_{d}=29.35±1.91 nM (FIGS. 1C and 15). As a positive control, the interaction of SapC with DOPS has a K_{d}=13.45±1.65 nM (FIGS. 1D and 16). There were no detectable interactions between GCase and DOPS, SapB-DOPS or SapC-DOPS, or between SapB and DOPS, under the same experimental conditions (FIG. 16). These data demonstrated that GCase has specific binding affinity for SapC-DOPS nanovesicles.

The number of SapC and GCase molecules in each DOPS nanovesicle is estimated based on the simple geometry and average size measurements for DOPS liposomes. It was estimated that each DOPS liposome nanoparticle contains 3.31×10⁵ DOPS molecules. In optimized SapC-DOPS-GCase formulation, about 75% GCase bound on SapC-DOPS, thus, there are approximately 3.68×10⁴ SapC and 3.95×10⁴ GCase molecules in each DOPS nanoparticle.

SapC contains fusogenic/binding (NH₂-region) and GCase activation (COOH-region) domains. To determine the formulation with maximal uptake of SapC-based DOPS nanovesicles formulated with GCase, SapC-DOPS to SapB-DOPS, and SapC mutant-DOPS were compared by measuring the changes in GCase activity in 4L;C* fibroblasts following incubation with these differing nanovesicles (FIG. 1E). Data from previous studies (not shown) indicate that mutant SapC (K13A) has intact GCase activation, but defective PS binding activity. Also, mutant SapC (Q48N) has no activation function for GCase, but has PS membrane fusogenic/binding activity and mutant SapC (K23A) has neither GCase activation nor PS binding activities. SapB, as a negative control, does not have fusogenic/binding or GCase activation function. These results and previous studies indicate that SapC in the DOPS nanovesicle retains its GCase activation function in addition to its fusogenic lipid binding activity, allowing it to potentially target PS exposed brain cells.

### Example 5. SapC-DOPS-GCase Formulation Preserves GCase Activity and Facilitates Cell Uptake

Serum stability of GCase in the SapC-DOPS-GCase formulation was determined by measuring GCase activity in WT mice sera (pH 7.4) or culture medium (pH 7.2, 10% FCS) at 0 to 8 hrs and compared to free GCase. The half-life of EC₅₀ values showed that SapC-DOPS-GCase is 3 times more stable then free GCase in mouse serum and 1.6 times more in the culture medium (FIGS. 8A-8C).

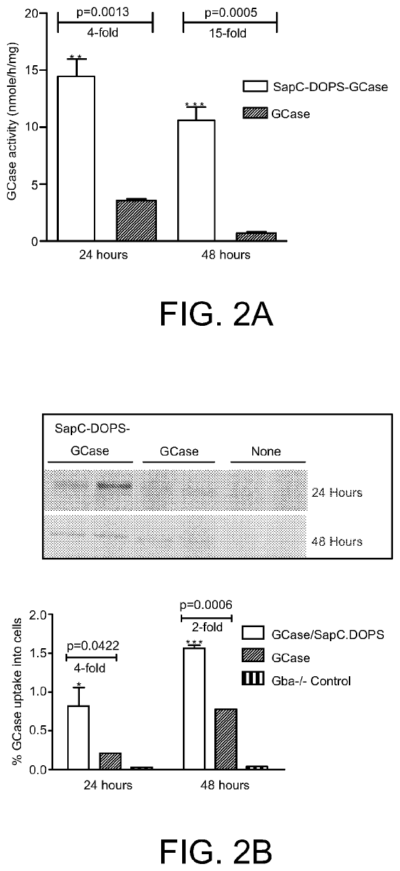

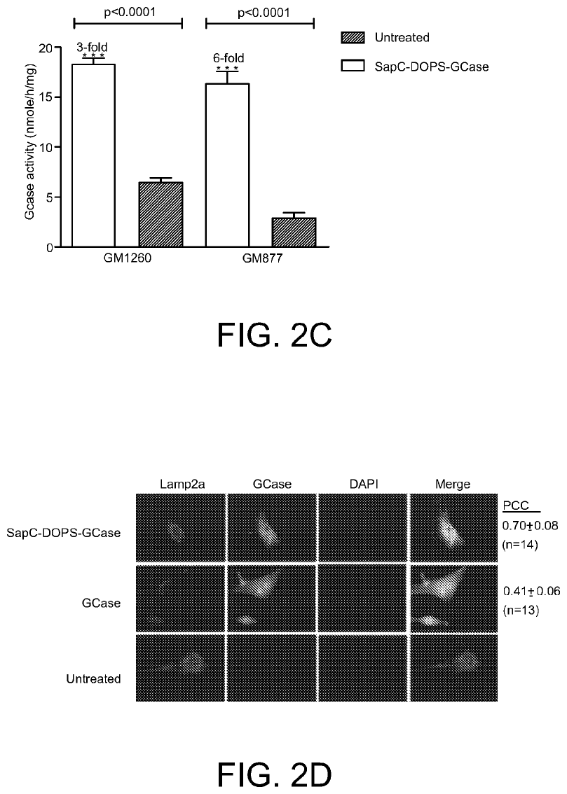

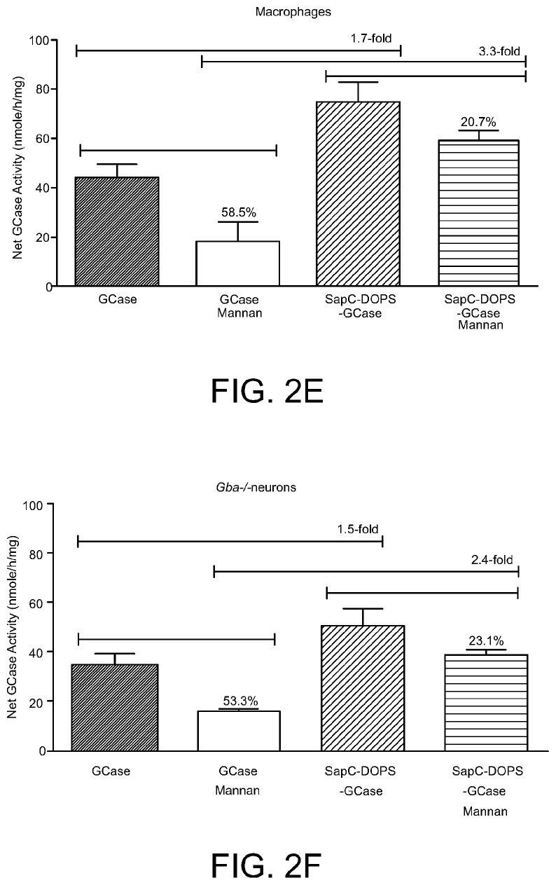

To determine whether SapC-DOPS improves GCase functionality, SapC-DOPS-GCase formulation [SapC/DOPS (1:9) mM with 80 μg GCase] was assayed for GCase activity and tested in mouse fibroblasts derived from Gba1^{-/-} newborn mice that have no functional GCase. Equal activities of SapC-DOPS-GCase and free GCase were added to media of Gba1^{-/-} fibroblasts. The cells with SapC-DOPS-GCase had 4-fold more GCase activity at 24 hrs and 15-fold more at 48 hrs than the cells incubated with free GCase (FIG. 2A). Cells incubated with SapC-DOPS-GCase showed higher total cellular GCase protein levels, determined by immunoblot, than those with free GCase at 24 hrs and 48 hrs after loading (FIG. 2B). Increased GCase activity and protein were detected 4 hrs after loading (data not shown). Human GD type 2 patient fibroblasts that have low levels of residual GCase activity incubated with SapC-DOPS-GCase also showed increase of GCase activity (FIG. 2C).

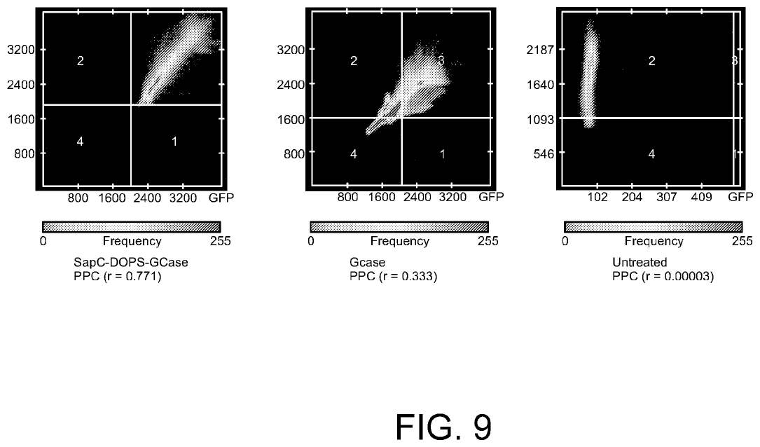

The SapC-DOPS-GCase taken up by the Gba1^{-/-} fibroblasts was targeted into lysosomes (FIG. 2D), whereas this was not obvious with free GCase. The co-localization of GCase with lysosomal marker, Lamp1, was determined by Pearson correlation coefficient (PCC) in the acquired image using Zeiss image analysis software (FIG. 9). The cells with SapC-DOPS-GCase showed higher PCC (0.70±0.08) compared to the cells with GCase (PCC=0.41±0.06), indicating more GCase targeted into the lysosome by SapC-DOPS-GCase. These results demonstrate that human GCase formulated with SapC-DOPS is targeted to, and is functional in GD fibroblasts. SapC-DOPS preserves GCase activity and protects GCase from denaturation, therefore facilitating cellular uptake and lysosomal localization.

### Example 6. Effect of Mannan on Uptake of SapC-DOPS-GCase

Oligo-mannosyl-terminated oligosaccharides on GCase (e.g., velaglucerase alfa) promote preferential uptake into myloid-lineage cells expressing the mannose receptor and neurons. To determine if cell uptake of SapC-DOPS-GCase is dependent on the mannose receptor, uptake was assessed with J774E macrophages which express this receptor and transformed mouse Gba1^{-/-} neurons in the presence or absence of mannan, a competitor for the mannose receptor. Both cell types were incubated with equal 8 U GCase, either as free GCase or as SapC-DOPS-GCase. The cells were washed thoroughly before the activity assay and confirmed by IF that there was no significant GCase binding on the cell surface. The cells with SapC-DOPS-GCase showed more GCase activity than with free GCase (FIGS. 2E and 2F). Mannan blocked ˜50% of free GCase uptake, but only ˜20% of SapC-DOPS-GCase in either macrophages or neurons (FIGS. 2E and 2F). These data suggest that SapC-DOPS-GCase enters the cells largely through routes other than the mannose receptor, e.g. direct cell membrane fusion.

### Example 7. Tissue Distribution and Brain Targeting of SapC-DOPS-GCase

As mentioned above, mannose-terminated oligosaccharides promote cellular uptake of GCase via the mannose receptor. However, several organs are poorly or inaccessible to these agents, including the brain, alveoli of the lungs, and lymph nodes. To assess the ability of SapC-DOPS-GCase to enter the brain and visceral tissues, 4L;C* mice were intravenously (i.v.) or intraperitoneally (i.p.) infused with equal amounts of protein (equal activity) of free GCase or SapC-DOPS-GCase. Their activities were kept constant in each experiment. In acute experiments, tissue GCase protein and activities were determined at 3 h, or post injection. Brain accessible SapC-DOPS-GCase was confirmed by immunoblot. Human GCase protein in the brain lysates was immunoprecipitated using specific rabbit anti-human GCase antibody (no cross-reactivity with mouse GCase) followed by immunoblotting with goat anti-human GCase (FIG. 3A). In comparison, human GCase protein was detected in the liver of free GCase and SapC-DOPS-GCase injected mice (FIG. 3E). SapC-DOPS-GCase was also delivered into WT mice brains, but at much lower levels than nGD mice (FIG. 3B). GCase protein in 4L;C* brains was detected up to the end point (24 h post-injection) of the experiment (FIG. 3C). Mice injected with SapC-DOPS-GCase showed GCase activity in the brain, but those injected with free GCase did not, indicating that SapC-DOPS allowed GCase access into the brain (FIG. 3D).

In visceral tissues, GCase activity was determined in SapC-DOPS-GCase i.p.-injected mice. Higher GCase activity was detected in the liver, spleen, lung, lymph nodes and bone marrow of SapC-DOPS-GCase injected mice than free GCase-injected mice suggesting that SapC-DOPS protects GCase from rapid clearance by the reticuloendothelial system. It also protects GCase from pH inactivation in serum as evidenced by the long half-life of 0.68 h in the serum (FIGS. 8A-8C). Liver lysate showed greater incorporation of SapC-DOPS-GCase than free GCase to liver (FIGS. 3E and 3F). By contrast, in comparison to free GCase injected tissues, SapC-DOPS-GCase-injected mice had increased GCase activity in lymph nodes and lung (FIG. 3F).

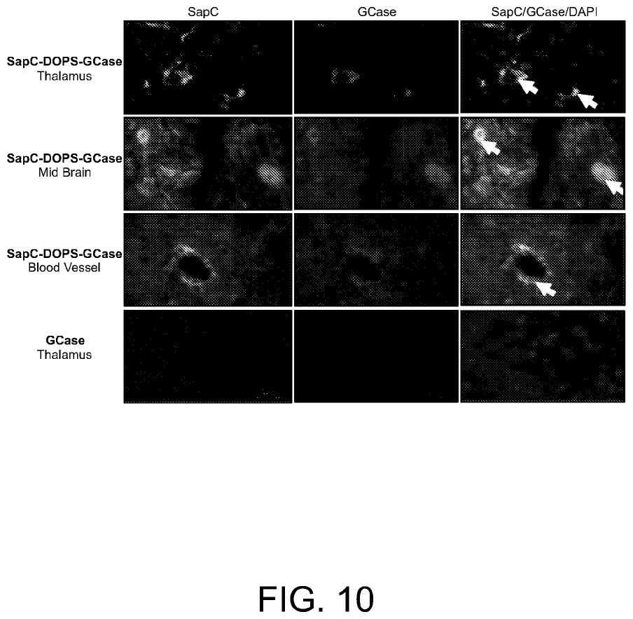

In SapC-DOPS-GCase-treated 4L;C* brains, GCase was observed lining the endothelium of blood vessels in the treated 4L;C* brain (FIG. 3G). GCase was trafficked into the lysosome in the brain cells of the 4L;C* mice that had been administered SapC-DOPS-GCase, but not free GCase (FIG. 3H). GCase signals were in »6% of neurons, 6-18% of astrocytes and 8-18% microglia cells counted in each brain region (FIGS. 3I and 3J). GCase target cells were distributed mainly in the cortex, midbrain, brain stem and thalamus (FIGS. 3J and 3K). GCase co-localized with SapC in treated mouse brain cells demonstrating that GCase is delivered by SapC-DOPS nanovesicles into the brain (FIG. 10). These results confirm that SapC-DOPS-GCase entered the brain cells when infused i.v., a clinically relevant, non-invasive procedure for GD treatment.

### Example 8. SapC-DOPS-GCase Mitigated Neuronal Phenotypes in nGD Mouse Model, 4L;C* Mice

The 4L;C* model is viable analog of human nGD that have progressive accumulation of substrates and CNS manifestations. 4L;C* mice has a Gba1 mutation V394L/V394L and lack of SapC. 4L;C* the mice used in this study are in the C57BL/6 J background with a median life span of 56 days. SapC-DOPS-GCase was administered to 4L;C* mice for evaluation of in vivo efficacy. The SapC-DOPS-GCase formulation used for mice injection was routinely assayed for GCase activity and a confirmed ˜80% GCase activity remained prior to use. In preliminary studies, SapC-DOPS-CVM and SapC-DOPS-GCase were delivered into young (13 day old) 4L;C* brains by i.p. injection. Before day 28, when the tail vein was not accessible, the formulation was administered by daily i.p. injections. Tail vein i.v. injection started at day 28 and 3 times per week. The treatment started when disease signs appeared around 21 days.

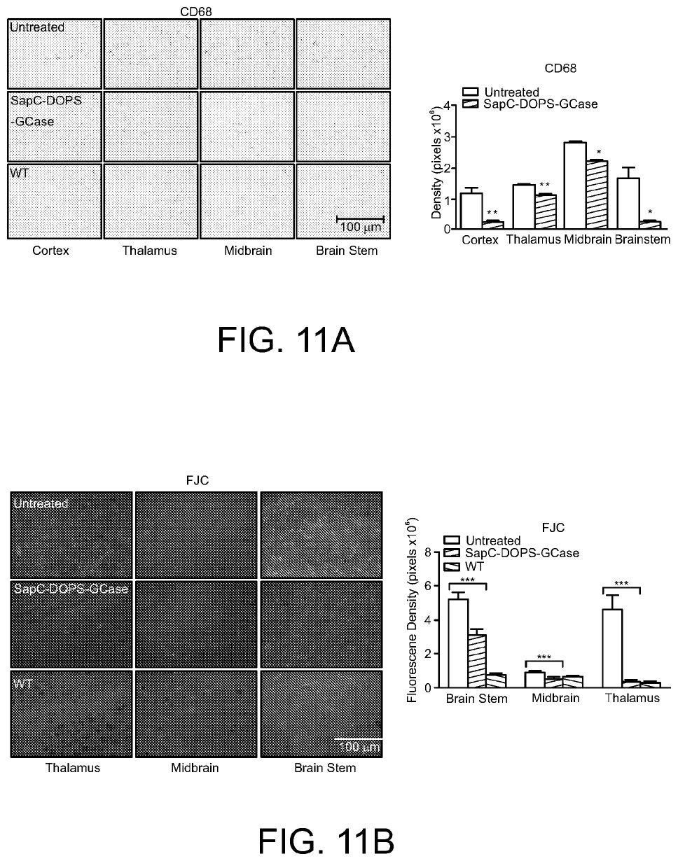

Treatment effect was evaluated in 4L;C* mice by neurological phenotype, inflammation, neurodegeneration, and substrate reduction. 4L;C* mice developed age-related hindlimb clasping behavior. 4L;WT littermates that have normal phenotype are used as controls to assess clasping behavior change in 4L;C* mice with treatment. The treated 4L;C* mice showed significantly delayed hindlimb clasping (FIG. 4A). 4L;C* mice developed hindlimb paralyses with abnormal gait, a sensorimotor function deficit. Compared to 4L;C* mice who received vehicle, SapC-DOPS-GCase treated 4L;C* mice had significantly improved stride length by gait analysis at 50 and 55 days of age (FIG. 4B). The SapC-DOPS-GCase treatment significantly prolonged 4L;C* survival to a median of 64 days vs. 56 days for untreated mice; a 14% extension compared to untreated 4L;C* or 15% compared to free GCase and Vehicle-4L;C* mice (FIG. 4C). Because free GCase does not cross the BBB, as expected, 4L;C* mice administered with free GCase did not have improved survival and compared to the untreated and vehicle groups (FIG. 4C). The treatment also reduced the substrate, glucosylceramide, accumulation in the brains of 4L;C* mice (FIG. 4D). Brain inflammation was analyzed by anti-CD68 staining on activated microglial cells and anti-GFAP staining on activated astrocytes. SapC-DOPS-GCase treatment significantly mitigated brain inflammation in 4L;C* mice in the cortex, thalamus, midbrain and brainstem regions (FIGS. 4E and 11A). Neurodegeneration, assayed by Fluoro-Jade C (FJC), an anionic fluorescent dye as a marker of degenerating neurons, was significantly diminished in SapC-DOPS-GCase treated 4L;C* brain stem, midbrain and thalamus regions (FIG. 11B). These data demonstrate improved neuronopathic phenotype in nGD mice by SapC-DOPS-GCase treatment.

### Example 9. SapC-DOPS In Vitro and In Vivo Efficacy

4L;C* mice have SapC deficiency so the efficacy of SapC-DOPS to replace SapC in 4L;C* mice was evaluated. Increased GCase activity in SapC-DOPS treated 4L;C* mouse fibroblasts indicates the uptake of SapC is functional (FIG. 1B). Compared to control mice, i.v. injected SapC-DOPS fluorescently labeled with CVM primarily localized to the thalamus of 4L;C* mice (FIG. 5A); this brain region exhibits inflammatory cells in this model. In the control, 4L;WT mice that do not show brain inflammation, low level of CVM was detected (FIG. 5A), suggesting the accessibility of SapC-DOPS into non-inflamed normal brain. To evaluate SapC-DOPS's in vivo efficacy, 4L;C* mice were treated with SapC-DOPS, which increased brain GCase activity (FIG. 5B) and survival (FIG. 5C). Both SapC-DOPS and SapC-DOPS-GCase improved abnormal gait at 45 days of age in 4L;C* mice (FIG. 5D). While SapC-DOPS alone had small effects on survival of 4L;C* mice, SapC-DOPS-GCase treated 4L;C* mice showed longer survival improved gait (FIGS. 5C and 5D), signifying enhanced efficacy when GCase was added to the SapC-DOPS nanovesicles.

### Example 10. Phosphatidylserine (PS)-Mediated Brain Targeting of SapC-DOPS Nanovesicles

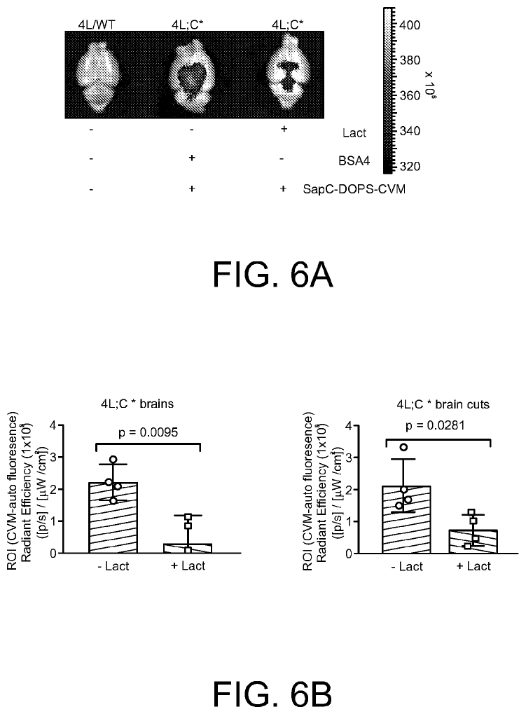

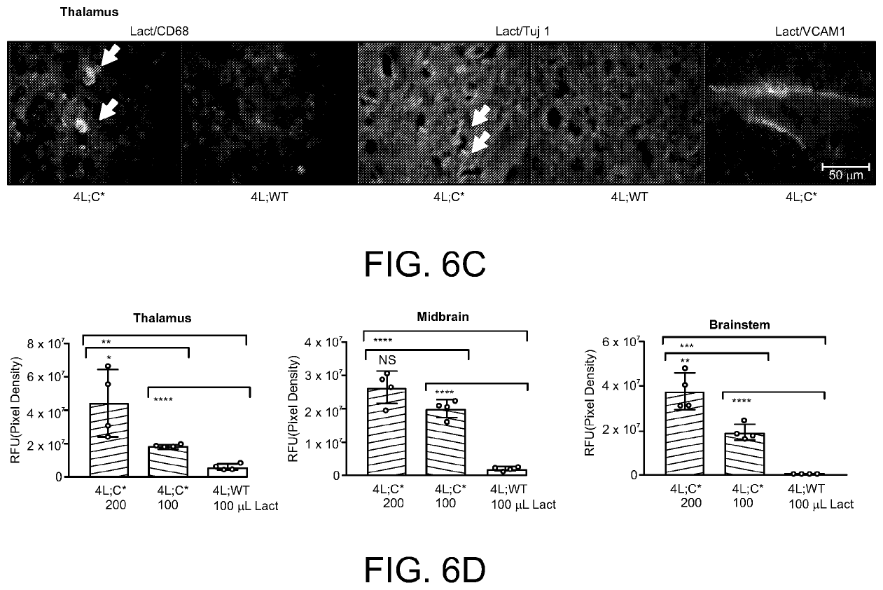

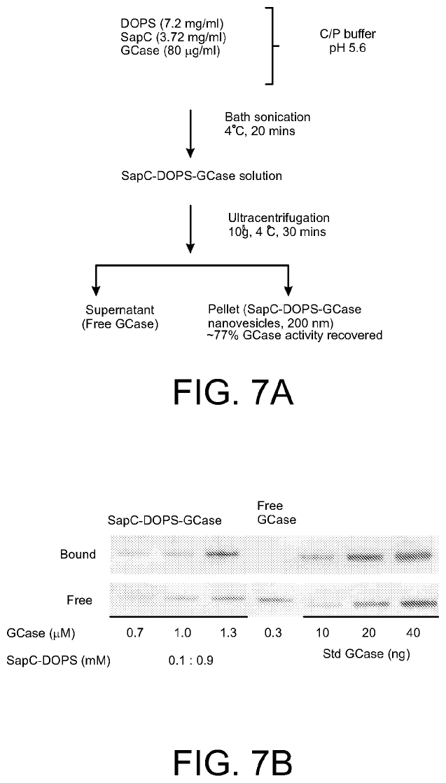

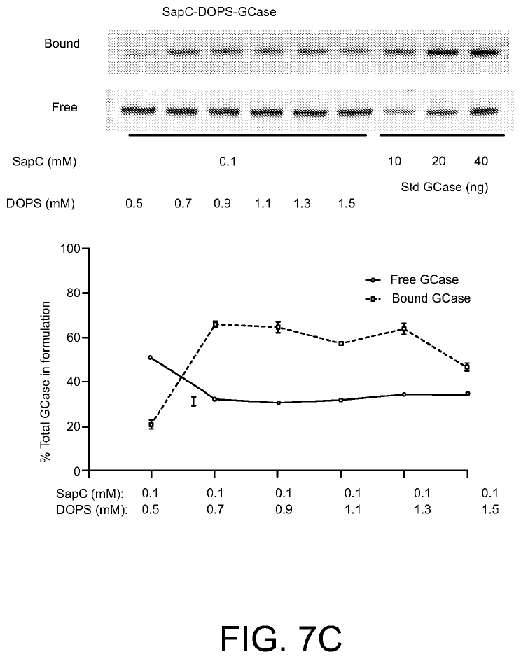

SapC, a membrane-associated protein, has a favorable binding affinity for unsaturated, negatively charged PS. To determine if SapC-DOPS targeting of 4L;C* brains is dependent on PS, 4L;C* mice were i.v. injected with lactadherin or BSA prior to SapC-DOPS-CVM. Lactadherin is a PS-specific binding protein that acts through its C-2 domain (Lact-C2). SapC and Lact-C2 both bind PS through its negatively charged head group. Lactadherin cannot enter intact cells so it binds PS only on the outside of the cells. It was previously demonstrated that lactadherin can block SapC-DOPS entry into glioblastoma (brain cancer) cells by shielding the cell surface PS, both in vitro and in vivo. The CVM signals were detected in the control, BSA injected 4L;C* mice, but were significantly reduced in lactadherin injected mice (FIGS. 6A and 6B). Brain cell types that lactadherin binds were determined in the 4L;C* mice i.v. administered with lactadherin-FITC. The lactadherin signals were on active microglial cells (CD68+), neurons (Tuj1+) and endothelium (VCAM1+) (FIG. 6C), but largely absent on astrocytes (GFAP+). Lactadherin signals were enriched in a dose-dependent manner in the inflammatory regions that have strong CD68 signals (FIG. 6D). To test whether lactadherin directly inhibits the PS-binding activity of SapC-DOPS nanovesicles, we treated SapC-DOPS-CVM with lactadherin at 37° C. for 30 mins, and collected the pellet after ultracentrifugation to remove free lactadherin. The mixture in the pellet showed the surface PS-binding activity on human brain tumor cells (Gli36) at the same level as the non-lactadherin treated SapC-DOPS-CVM by flow cytometry analysis (FIG. 12). This indicates that free lactadherin cannot significantly reduce the PS-binding effect of SapC-DOPS-CVM. These data support a PS mediated pathway via BBB for SapC-DOPS targeting into 4L;C* brain and especially inflamed regions.

### Example 11. Lymphatic Pathway is Involved in SapC-Based Nanovesicles Targeting to CNS

To determine the pathway of SapC-DOPS targeting to CNS, SapC-DOPS-CVM was i.v. injected to WT mice. The CVM had a different distribution in brain meninges compared to lectin-stained blood vessels (FIG. 13A), indicating SapC-DOPS does not accumulate in these blood vessels. However, GCase was detected in mouse brain meninges and co-localized with LYVE1 (lymphatic vessel marker) in 4L;C* mice 24 h after i.v. administration of SapC-DOPS-GCase (FIG. 13B). CVM infiltrated into lymph nodes in mice i.v. administered SapC-DOPSCVM (FIG. 13C). Lymphatics-defective transgenic mice, K14-VEGFR3-Ig, develop a lymphedema-like phenotype characterized by edema, swelling of feet and dermal fibrosis but they survive to adulthood despite the lack of lymphatic vessels in several tissues. There is regeneration of the lymphatic vasculature in some tissues but not in the brain where microlymphatics are absent in adult K14-VEGFR3-Ig mice. To test whether the vesicle accumulation in brain is based on the meninges-mediated transport mechanism, we generated orthotopic brain tumors (10 £ 105 LLC-GFP cells) in K14-VEGFR3-Ig mice. Long-term SapC-DOPS accumulation was dramatically reduced in brain tumors of these mice (FIG. 13D). The data indicate that the meningeal-lymphatics are required for SapC-based vesicle uptake in diseased brains and suggest that SapC-based nanovesicles target the CNS through the blood-lymphatic loop.

### Example 12. SapC-DOPS-GCase Formulation

Determination of the composition of SapC-DOPS and the stoichiometry of GCase versus SapC-DOPS in the SapC-DOPS-GCase formulation was tested in two ways: 1) Various GCase amounts (0.7-1.3 μM) versus fixed ratios of SapC to DOPS (0.1 to 0.9 mM). 2) Fixed amount of SapC (0.1 mM) to various DOPS (0.5 to 1.1 mM) in the formulation with 1.3 μM GCase (1 U=10 μg of Velaglucerase alfa, GCase). SapC and DOPS were mixed at the indicated ratio (Tables 1 and 2 (FIG. 15)) in 250 μL CP buffer (pH 5.6) with GCase. The formulation mixture was bath sonicated for 15 mins at 4° C., diluted to 1 mL with CP buffer and centrifuged at 100,000×g for 30 mins at 4° C. in an ultra-centrifuge (Beckman, MA). Supernatants (1 mL) from each formulated mixture contain free GCase. The pellet with GCase bound to SapC-DOPS was resuspended into 250 μL CP buffer by sonication at 4° C. The activity and amount of free and bound GCase were determined by a GCase activity assay and immunoblots, respectively. The density of each band on the immunoblot was quantitated using GCase, in gradient amount, as a standard curve (linear regression fit) on the right panel of the same gel. Then the loaded samples (based on their concentration and loading volume) were back-calculated to total volume of bound or free to get the total GCase in either bound or free form after adjusted with dilution factor, and presents as % of bound GCase and % of free GCase in Tables 1 and 2 (FIG. 15).

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

What is claimed is:

1. A method of treating a subject suffering from Gaucher Disease, the method comprising administering to the subject an effective amount of a composition comprising:
saposin C (SapC) and dioleoylphosphatidylserine (SapC-DOPS); and
acid β-glucosidase (GCase),
wherein SapC, DOPS, and GCase are present in the composition in a ratio of about 0.1 mM SapC:about 0.7 mM to about 0.9 mM DOPS:about 1.3 KM GCase.

2. The method according to claim 1, wherein the composition comprises SapC-DOPS-GCase nanovesicles.

3. The method according to claim 2, wherein a molar ratio of saposin C (SapC) to dioleoylphosphatidylserine (DOPS) in the SapC-DOPS-GCase nanovesicles is about 1:7.

4. The method according to claim 2, wherein the GCase is bound to SapC in the presence of DOPS via non-covalent interactions selected from the group consisting of hydrophobic interactions, van der Waals forces, and combinations thereof.

5. (canceled)

6. The method according to claim 1, wherein the composition is administered intravenously.

7. The method according to claim 1, wherein the composition crosses a blood brain barrier of the subject.

8. The method according to claim 1, wherein Gaucher Disease comprises neuronopathic Gaucher Disease (nGD).

9. The method according to claim 8, wherein the nGD is selected from the group consisting of Gaucher Disease type 2 and Gaucher Disease type 3.

10. The method according to claim 1, further comprising administering to the subject a second therapeutic agent effective for the treatment of Gaucher Disease.

11. The method according to claim 10, wherein the second therapeutic agent is selected from an enzyme replacement therapy, a substrate reduction therapy, and a pharmacological chaperone.

12. The method according to claim 11, wherein the second therapeutic agent is an enzyme replacement therapy selected from the group consisting of imiglucerase, velaglucerase alfa, taliglucerase alfa, and combinations thereof.

13. The method according to claim 11, wherein the second therapeutic agent is a substrate reduction therapy selected from the group consisting of miglustat, eliglustat, venglustat, and combinations thereof.

14. The method according to claim 11, wherein the second therapeutic agent is a pharmacological chaperone selected from the group consisting of ambroxol hydrochloride, N-(n-nonyl)deoxynojirimycin (NN-DNJ), and combinations thereof.

15. A pharmaceutical composition comprising:
a nanovesicle comprising saposin C (SapC), dioleoylphosphatidylserine (SapC-DOPS), and acid β-glucosidase (GCase); and
a pharmaceutically-acceptable carrier,
wherein SapC, DOPS, and GCase are present in the composition in a ratio of about 0.1 mM SapC:about 0.7 mM to about 0.9 mM DOPS:about 1.3 μM GCase.

16. The pharmaceutical composition according to claim 15, wherein saposin C (SapC) and dioleoylphosphatidylserine (DOPS) are present in a molar ratio of about 1:7.

17. (canceled)

18. The pharmaceutical composition according to claim 15, wherein the GCase is bound to SapC in the presence of DOPS via non-covalent interactions selected from the group consisting of hydrophobic interactions, van der Waals forces, and combinations thereof.

19. The pharmaceutical composition according to claim 15, formulated for intravenous administration.

20. A nanovesicle comprising saposin C (SapC), dioleoylphosphatidylserine (DOPS), and acid β-glucosidase (GCase).

21. The nanovesicle according to claim 20, wherein a molar ratio of SapC to DOPS is about 1:7.

22. The nanovesicle according to claim 20, wherein the GCase is bound to SapC in the presence of DOPS via non-covalent interactions selected from the group consisting of hydrophobic interactions, van der Waals forces, and combinations thereof.
